SEQUENCE LISTING (140 sequences)

SEQ ID NO: 1 - protein, 6 aa, synthetic construct
  REGION 1..6: note = Synthetic Construct
YMAPEV

SEQ ID NO: 2 - protein, 6 aa, synthetic construct
  REGION 1..6: note = Synthetic Construct
IIAPEG

SEQ ID NO: 3 - protein, 6 aa, synthetic construct
  REGION 1..6: note = Synthetic Construct
EIAEAL

SEQ ID NO: 4 - protein, 6 aa, synthetic construct
  REGION 1..6: note = Synthetic Construct
SNVAEA

SEQ ID NO: 5 - protein, 6 aa, synthetic construct
  REGION 1..6: note = Synthetic Construct
ANIAEA

SEQ ID NO: 6 - protein, 6 aa, synthetic construct
  REGION 1..6: note = Synthetic Construct
ANMAEN

SEQ ID NO: 7 - protein, 6 aa, synthetic construct
  REGION 1..6: note = Synthetic Construct
NWAENA

SEQ ID NO: 8 - protein, 6 aa, synthetic construct
  REGION 1..6: note = Synthetic Construct
LWAEAK

SEQ ID NO: 9 - protein, 6 aa, synthetic construct
  REGION 1..6: note = Synthetic Construct
LIAEAK

SEQ ID NO: 10 - protein, 6 aa, synthetic construct
  REGION 1..6: note = Synthetic Construct
NVAENA

SEQ ID NO: 11 - protein, 6 aa, synthetic construct
  REGION 1..6: note = Synthetic Construct
LVAEAH

SEQ ID NO: 12 - protein, 6 aa, synthetic construct
  REGION 1..6: note = Synthetic Construct
LIANAK

SEQ ID NO: 13 - protein, 6 aa, synthetic construct
  REGION 1..6: note = Synthetic Construct
  SITE 5: note = D-glutamate
LFAPEA

SEQ ID NO: 14 - protein, 6 aa, synthetic construct
  REGION 1..6: note = Synthetic Construct
  SITE 5: note = D-glutamate
LIAPEA

SEQ ID NO: 15 - protein, 6 aa, synthetic construct
  REGION 1..6: note = Synthetic Construct
TNWAEN

SEQ ID NO: 16 - protein, 6 aa, synthetic construct
  REGION 1..6: note = Synthetic Construct
TWAPES

SEQ ID NO: 17 - protein, 6 aa, synthetic construct
  REGION 1..6: note = Synthetic Construct
TWSPEL

SEQ ID NO: 18 - protein, 6 aa, synthetic construct
  REGION 1..6: note = Synthetic Construct
  MOD_RES 1: note = Xaa = Sarcosine
  MOD_RES 6: note = Xaa = N-methyl alanine
XWAEAX

SEQ ID NO: 19 - protein, 18 aa, synthetic construct
  REGION 1..18: note = Synthetic Construct
  MOD_RES 1: note = Xaa = Sarcosine
  SITE 4: note = D-glutamate
  MOD_RES 6: note = Xaa = Sarcosine
XAATRPALAGLUALAXAA

SEQ ID NO: 20 - protein, 6 aa, synthetic construct
  REGION 1..6: note = Synthetic Construct
AWAEAK

SEQ ID NO: 21 - protein, 6 aa, synthetic construct
  REGION 1..6: note = Synthetic Construct
  SITE 4: note = D-glutamate
AWAEAK

SEQ ID NO: 22 - protein, 6 aa, synthetic construct
  REGION 1..6: note = Synthetic Construct
AWANAK

SEQ ID NO: 23 - protein, 6 aa, synthetic construct
  REGION 1..6: note = Synthetic Construct
  SITE 4: note = D-Asparagine
AWANAK

SEQ ID NO: 24 - protein, 6 aa, synthetic construct
  REGION 1..6: note = Synthetic Construct
AWAPEA

SEQ ID NO: 25 - protein, 6 aa, synthetic construct
  REGION 1..6: note = Synthetic Construct
  SITE 5: note = D-glutamate
AWAPEA

SEQ ID NO: 26 - protein, 6 aa, synthetic construct
  REGION 1..6: note = Synthetic Construct
PSAPEN

SEQ ID NO: 27 - protein, 6 aa, synthetic construct
  REGION 1..6: note = Synthetic Construct
HMAPEV

SEQ ID NO: 28 - protein, 6 aa, synthetic construct
  REGION 1..6: note = Synthetic Construct
YIAPEV

SEQ ID NO: 29 - protein, 6 aa, synthetic construct
  REGION 1..6: note = Synthetic Construct
KAPEPL

SEQ ID NO: 30 - protein, 6 aa, synthetic construct
  REGION 1..6: note = Synthetic Construct
LVAEAK

SEQ ID NO: 31 - protein, 6 aa, synthetic construct
  REGION 1..6: note = Synthetic Construct
WMAPET

SEQ ID NO: 32 - protein, 6 aa, synthetic construct
  REGION 1..6: note = Synthetic Construct
EAPEDL

SEQ ID NO: 33 - protein, 6 aa, synthetic construct
  REGION 1..6: note = Synthetic Construct
DVAPED

SEQ ID NO: 34 - protein, 6 aa, synthetic construct
  REGION 1..6: note = Synthetic Construct
YLAPEV

SEQ ID NO: 35 - protein, 6 aa, synthetic construct
  REGION 1..6: note = Synthetic Construct
YMAPEH

SEQ ID NO: 36 - protein, 6 aa, synthetic construct
  REGION 1..6: note = Synthetic Construct
QIAEGM

SEQ ID NO: 37 - protein, 6 aa, synthetic construct
  REGION 1..6: note = Synthetic Construct
WTAPEA

SEQ ID NO: 38 - protein, 6 aa, synthetic construct
  REGION 1..6: note = Synthetic Construct
WYAPEC

SEQ ID NO: 39 - protein, 6 aa, synthetic construct
  REGION 1..6: note = Synthetic Construct
SNVAEA

SEQ ID NO: 40 - protein, 6 aa, synthetic construct
  REGION 1..6: note = Synthetic Construct
YRAPEI

SEQ ID NO: 41 - protein, 6 aa, synthetic construct
  REGION 1..6: note = Synthetic Construct
ANVAEK

SEQ ID NO: 42 - protein, 7 aa, synthetic construct
  REGION 1..7: note = Synthetic Construct
ANVAENA

SEQ ID NO: 43 - protein, 7 aa, synthetic construct
  REGION 1..7: note = Synthetic Construct
GGQIANI

SEQ ID NO: 44 - protein, 6 aa, synthetic construct
  REGION 1..6: note = Synthetic Construct
  MOD_RES 2: note = Xaa = Aminoisobutyric acid
IXAEAK

SEQ ID NO: 45 - protein, 6 aa, synthetic construct
  REGION 1..6: note = Synthetic Construct
  MOD_RES 3: note = Xaa = N-methyl alanine
IIXEAK

SEQ ID NO: 46 - protein, 8 aa, synthetic construct
  REGION 1..8: note = Synthetic Construct
  MOD_RES 7: note = Xaa = beta-alanine
IWGLDGXK

SEQ ID NO: 47 - protein, 6 aa, synthetic construct
  REGION 1..6: note = Synthetic Construct
  MOD_RES 3: note = Xaa = N-methyl alanine
IIXEAK

SEQ ID NO: 48 - protein, 6 aa, synthetic construct
  REGION 1..6: note = Synthetic Construct
  MOD_RES 2: note = Xaa = aminobutyric acid
LXAEAK

SEQ ID NO: 49 - protein, 6 aa, synthetic construct
  REGION 1..6: note = Synthetic Construct
  MOD_RES 2: note = Xaa = Aminoisobutyric acid
LXAEAK

SEQ ID NO: 50 - protein, 6 aa, synthetic construct
  REGION 1..6: note = Synthetic Construct
  SITE 3: note = D-alanine
LIAEAK

SEQ ID NO: 51 - protein, 6 aa, synthetic construct
  REGION 1..6: note = Synthetic Construct
  MOD_RES 3: note = Xaa = N-methyl alanine
LIXEAK

SEQ ID NO: 52 - protein, 6 aa, synthetic construct
  REGION 1..6: note = Synthetic Construct
  MOD_RES 4: note = Xaa = Aminoisobutyric acid
LIAXEA

SEQ ID NO: 53 - protein, 6 aa, synthetic construct
  REGION 1..6: note = Synthetic Construct
  SITE 4: note = D-glutamate
LIAEAK

SEQ ID NO: 54 - protein, 7 aa, synthetic construct
  REGION 1..7: note = Synthetic Construct
LIADEAK

SEQ ID NO: 55 - protein, 6 aa, synthetic construct
  REGION 1..6: note = Synthetic Construct
LIAEAA

SEQ ID NO: 56 - protein, 6 aa, synthetic construct
  REGION 1..6: note = Synthetic Construct
LIAEAK

SEQ ID NO: 57 - protein, 6 aa, synthetic construct
  REGION 1..6: note = Synthetic Construct
LIAGEG

SEQ ID NO: 58 - protein, 6 aa, synthetic construct
  REGION 1..6: note = Synthetic Construct
LIAGGE

SEQ ID NO: 59 - protein, 6 aa, synthetic construct
  REGION 1..6: note = Synthetic Construct
LIAKGK

SEQ ID NO: 60 - protein, 6 aa, synthetic construct
  REGION 1..6: note = Synthetic Construct
LIANAK

SEQ ID NO: 61 - protein, 6 aa, synthetic construct
  REGION 1..6: note = Synthetic Construct
LIAPEA

SEQ ID NO: 62 - protein, 6 aa, synthetic construct
  REGION 1..6: note = Synthetic Construct
  MOD_RES 5: note = Xaa = Hydroxyproline
LIAPXA

SEQ ID NO: 63 - protein, 6 aa, synthetic construct
  REGION 1..6: note = Synthetic Construct
LIAQAK

SEQ ID NO: 64 - protein, 8 aa, synthetic construct
  REGION 1..8: note = Synthetic Construct
  MOD_RES 7: note = Xaa = beta-alanine
IWGLDGXK

SEQ ID NO: 65 - protein, 6 aa, synthetic construct
  REGION 1..6: note = Synthetic Construct
  MOD_RES 4: note = Xaa = Aminoisobutyric acid
LIAXEA

SEQ ID NO: 66 - protein, 7 aa, synthetic construct
  REGION 1..7: note = Synthetic Construct
  MOD_RES 5: note = Xaa = D-glutamate
LIAPXAK

SEQ ID NO: 67 - protein, 6 aa, synthetic construct
  REGION 1..6: note = Synthetic Construct
  MOD_RES 3: note = Xaa = Hydroxyproline
LIXEAK

SEQ ID NO: 68 - protein, 8 aa, synthetic construct
  REGION 1..8: note = Synthetic Construct
  MOD_RES 7: note = Xaa = beta-alanine
WIALEGXK

SEQ ID NO: 69 - protein, 6 aa, synthetic construct
  REGION 1..6: note = Synthetic Construct
  SITE 5: note = D-glutamate
FIAPEA

SEQ ID NO: 70 - protein, 7 aa, synthetic construct
  REGION 1..7: note = Synthetic Construct
ANVAENA

SEQ ID NO: 71 - protein, 6 aa, synthetic construct
  REGION 1..6: note = Synthetic Construct
LIAEAK

SEQ ID NO: 72 - protein, 6 aa, synthetic construct
  REGION 1..6: note = Synthetic Construct
LIAPEA

SEQ ID NO: 73 - protein, 6 aa, synthetic construct
  REGION 1..6: note = Synthetic Construct
  MOD_RES 2: note = Xaa = alpha-amino isobutyric acid
LXAEAK

SEQ ID NO: 74 - protein, 6 aa, synthetic construct
  REGION 1..6: note = Synthetic Construct
  MOD_RES 3: note = Xaa = N-methyl alanine
LIXEAK

SEQ ID NO: 75 - protein, 6 aa, synthetic construct
  REGION 1..6: note = Synthetic Construct
  MOD_RES 2: note = Xaa = alpha-amino butyric acid
LXAEAK

SEQ ID NO: 76 - protein, 7 aa, synthetic construct
  REGION 1..7: note = Synthetic Construct
GGQIANI

SEQ ID NO: 77 - protein, 6 aa, synthetic construct
  REGION 1..6: note = Synthetic Construct
EGIAGK

SEQ ID NO: 78 - protein, 6 aa, synthetic construct
  REGION 1..6: note = Synthetic Construct
LIADAK

SEQ ID NO: 79 - protein, 6 aa, synthetic construct
  REGION 1..6: note = Synthetic Construct
LIANAK

SEQ ID NO: 80 - protein, 6 aa, synthetic construct
  REGION 1..6: note = Synthetic Construct
LIAEAA

SEQ ID NO: 81 - protein, 6 aa, synthetic construct
  REGION 1..6: note = Synthetic Construct
LIAQAK

SEQ ID NO: 82 - protein, 6 aa, synthetic construct
  REGION 1..6: note = Synthetic Construct
LIAGGE

SEQ ID NO: 83 - protein, 6 aa, synthetic construct
  REGION 1..6: note = Synthetic Construct
LIAGEG

SEQ ID NO: 84 - protein, 6 aa, synthetic construct
  REGION 1..6: note = Synthetic Construct
ANVAEK

SEQ ID NO: 85 - protein, 6 aa, synthetic construct
  REGION 1..6: note = Synthetic Construct
LIAKGK

SEQ ID NO: 86 - protein, 15 aa, synthetic construct
  REGION 1..15: note = Synthetic Construct
GTPGPQGIAGQRGVV

SEQ ID NO: 87 - protein, 6 aa, synthetic construct
  REGION 1..6: note = Synthetic Construct
  MOD_RES 2: note = Xaa = alpha-amino isobutyric acid
IXAEAK

SEQ ID NO: 88 - protein, 6 aa, synthetic construct
  REGION 1..6: note = Synthetic Construct
  MOD_RES 2: note = Xaa = alpha-amino butyric acid
LXAEAK

SEQ ID NO: 89 - protein, 6 aa, synthetic construct
  REGION 1..6: note = Synthetic Construct
LPAEAK

SEQ ID NO: 90 - protein, 6 aa, synthetic construct
  REGION 1..6: note = Synthetic Construct
LIPEAK

SEQ ID NO: 91 - protein, 6 aa, synthetic construct
  REGION 1..6: note = Synthetic Construct
  MOD_RES 3: note = Xaa = alpha-amino isobutyric acid
LIXEAK

SEQ ID NO: 92 - protein, 6 aa, synthetic construct
  REGION 1..6: note = Synthetic Construct
  MOD_RES 4: note = Xaa = alpha-amino isobutyric acid
LIAXEA

SEQ ID NO: 93 - protein, 8 aa, synthetic construct
  REGION 1..8: note = Synthetic Construct
  MOD_RES 7: note = Xaa = b-Alanine
IWGLDGXK

SEQ ID NO: 94 - protein, 8 aa, synthetic construct
  REGION 1..8: note = Synthetic Construct
  MOD_RES 7: note = Xaa = b-Alanine
WIALEGXK

SEQ ID NO: 95 - protein, 9 aa, synthetic construct
  REGION 1..9: note = Synthetic Construct
GPQGIAGQR

SEQ ID NO: 96 - protein, 6 aa, synthetic construct
  REGION 1..6: note = Synthetic Construct
QGIAGQ

SEQ ID NO: 97 - protein, 7 aa, synthetic construct
  REGION 1..7: note = Synthetic Construct
QGIAGQR

SEQ ID NO: 98 - protein, 6 aa, synthetic construct
  REGION 1..6: note = Synthetic Construct
FGIAGF

SEQ ID NO: 99 - protein, 5 aa, synthetic construct
  REGION 1..5: note = Synthetic Construct
GIAGQ

SEQ ID NO: 100 - protein, 6 aa, synthetic construct
  REGION 1..6: note = Synthetic Construct
QGAIAQ

SEQ ID NO: 101 - protein, 6 aa, synthetic construct
  REGION 1..6: note = Synthetic Construct
FGIAGF

SEQ ID NO: 102 - protein, 6 aa, synthetic construct
  REGION 1..6: note = Synthetic Construct
CGIAGC

SEQ ID NO: 103 - protein, 6 aa, synthetic construct
  REGION 1..6: note = Synthetic Construct
EGIAGK

SEQ ID NO: 104-106 - skipped (3 entries)

SEQ ID NO: 107 - protein, 6 aa, synthetic construct
  REGION 1..6: note = Synthetic Construct
  MOD_RES 3: note = Xaa = N-methyl alanine
IIXEAK

SEQ ID NO: 108 - protein, 6 aa, synthetic construct
  REGION 1..6: note = Synthetic Construct
  SITE 3: note = D-alanine
LIAEAK

SEQ ID NO: 109 - protein, 6 aa, synthetic construct
  REGION 1..6: note = Synthetic Construct
  SITE 4: note = Xaa = D-glutamic acid
LIAXAK

SEQ ID NO: 110 - protein, 6 aa, synthetic construct
  REGION 1..6: note = Synthetic Construct
  VARIANT 5: note = Xaa = Any Amino Acid or D-glutamic acid
LIAPXA

SEQ ID NO: 111 - protein, 6 aa, synthetic construct
  REGION 1..6: note = Synthetic Construct
  VARIANT 4: note = Xaa = Any Amino Acid or trans-4-hydroxyproline
LIAXAK

SEQ ID NO: 112 - protein, 4 aa, synthetic construct
  REGION 1..4: note = Synthetic Construct
LIAE

SEQ ID NO: 113 - protein, 4 aa, synthetic construct
  REGION 1..4: note = Synthetic Construct
NVAE

SEQ ID NO: 114 - protein, 6 aa, synthetic construct
  REGION 1..6: note = Synthetic Construct
  SITE 4: note = D-glutamate
NVAENA

SEQ ID NO: 115 - protein, 7 aa, synthetic construct
  REGION 1..7: note = Synthetic Construct
  SITE 5: note = D-glutamate
ANVAENA

SEQ ID NO: 116 - protein, 6 aa, synthetic construct
  REGION 1..6: note = Synthetic Construct
  SITE 5: note = D-glutamate
YMAPEV

SEQ ID NO: 117-140 - skipped (24 entries)

(19) United States
(12) Patent Application Publication
(10) Pub. No.: US 2024/0180997 A1
(43) Pub. Date: Jun. 6, 2024

(54) PEPTIDE INHIBITORS OF NF KAPPA B AND USE THEREOF IN TREATMENT OF COVID-19 AND INFLAMMATORY DISEASES

(71) Applicant: Rajendra Sahai BHATNAGAR, Burlingame, CA (US)

(72) Inventor: Rajendra Sahai BHATNAGAR, Burlingame, CA (US)

(21) Appl. No.: 18/386,971

(22) Filed: Nov. 3, 2023

Related U.S. Application Data
(63) Continuation of application No. 17/459,842, filed on Aug. 27, 2021, now abandoned.
(60) Provisional application No. 63/072,025, filed on Aug. 28, 2020.

(51) Int. Cl. A61K 38/08; A61P 31/14

(52) U.S. Cl. CPC A61K 38/08; A61P 31/14

(57) ABSTRACT

Peptides and methods of use thereof, are disclosed for use in treating disorders caused by the novel coronavirus SARS-COV-2 or resulting from COVID-19 disease, such as systemic inflammation or pneumonia. The peptides modulate the activity of the transcription factor NF κB.

### CROSS REFERENCE TO RELATED APPLICATIONS

This application claims priority benefit of U.S. Provisional Patent Application No. 63/072,025, filed Aug. 28, 2020. The contents of that application are hereby incorporated by reference herein in their entirety.

### FIELD OF THE INVENTION

Peptides are disclosed which can be used to treat symptoms and disorders arising from infection with SARS-COV-2, the pathogen responsible for COVID-19 disease, as well as inflammation and immune dysregulation caused by other pathogens or diseases.

### TECHNICAL FIELD

The present disclosure relates to the use of the disclosed compounds for use in prevention and therapy of COVID-19 disease symptoms, including pneumonia and the sequelae of pneumonia and systemic inflammation, caused by infection with the novel coronavirus SARS-COV-2.

### BACKGROUND

NF κB is a contributory factor in a large number of diseases and disorders. It is involved in numerous pathways mediating cell proliferation, survival, apoptosis, adhesion, invasion, and neo-vascularization in various cell types. Peptides which inhibit NF κB have been described in WO 2016/049580 and U.S. Pat. No. 10,030,048.

COVID-19, the disease caused by the novel coronavirus SARS-COV-2 (also referred to as 2019-nCOV) first identified in 2019, has resulted in very significant morbidity and mortality, as well as very serious world-wide social and economic disruption. Common symptoms arising in COVID-19 disease include lesions in the lungs and pneumonia, which can progress to respiratory failure and death.

Infection with SARS-COV-2 results in an immune response to attack the invading microorganism. Immune cells are summoned to the site of infection, and defensive molecules are secreted. However, the immune system can react so strongly that the immune reaction damages the infected tissue, such as by overproduction of cytokines and metalloproteinases. An overactive immune system can cause edema and fibrosis in lungs; this walls off the intruder, but reduces lung capacity. The immune system overreaction can have deleterious systemic effects as well, in a so-called “cytokine storm,” also known as hypercytokinemia. Several inflammation can develop in several tissues. This uncontrolled immune response can lead to damage in multiple systems, such as cardiac, vascular, hepatic, and renal damage. While younger patients appear less vulnerable to COVID-19 than older patients, they may develop a serious condition called multisystem inflammatory syndrome in children (MIS-C).

Therapeutic agents are urgently needed to alleviate the severe consequences of COVID-19. The current disclosure describes the use of peptide inhibitors of NF kappa B for use in treatment of COVID-19 and its associated symptoms.

### SUMMARY OF THE INVENTION

Compositions and methods for inhibiting the activity or the activation of NF κB are disclosed herein. Compounds which are four to twenty residue peptides, such as six to twenty residue peptides, are disclosed herein. The disclosed peptides inhibit the activation of NF κB and differentially modulate genes affected by NF κB. Methods for the use of the disclosed peptides in the treatment of diseases and disorders in humans and non-human patients in need of such treatments is also provided. Additional features and advantages of the disclosure will be apparent from the detailed description which follows, taken in conjunction with the accompanying drawings, which together illustrate, by way of example, features of the disclosure.

In any of the embodiments herein, any of the groups of peptides, or any of the groups of peptides indicated as usable for treatment of a disease or disorder, can additionally carry the proviso that the peptide contains at least one non-naturally-occurring amino acid. In any of the embodiments herein, any of the groups of peptides, or any of the groups of peptides indicated as usable for treatment of a disease or disorder, can additionally carry the proviso that the peptide contains at least one non-proteinogenic amino acid. In any of the embodiments herein, any of the groups of peptides, or any of the groups of peptides indicated as usable for treatment of a disease or disorder, can additionally carry the proviso that the peptide contains at least one D-amino acid.

In one embodiment, disclosed herein is the peptide YMAP[D]EV (L-tyrosyl-L-methionyl-L-alanyl-L-prolyl-D-glutamyl-L-valine) and its use in treating COVID-19. In one embodiment, disclosed herein is the peptide ANVAENA (L-alanyl-L-asparaginyl-L-valyl-L-alanyl-L-glutamyl-L-asparaginyl-L-alanine) and its use in treating COVID-19. In one embodiment, disclosed herein is the combination of the peptides YMAP[D]EV and ANVAENA, and the use of the combination in treating COVID-19.

In one embodiment, the peptides are selected from the group consisting of peptides of four to twenty amino acid residues in length, comprising the sequence:
Xxx-Ala-Pro-Glu
   where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (1). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113. Peptides of four to twenty amino acid residues in length comprising CORE SEQUENCE (1) are designated as the peptides of CORE SEQUENCE (1).

In one embodiment of the peptides of CORE SEQUENCE (1), the peptides are selected from the group consisting of peptides of four to twenty amino acid residues in length, comprising the sequence:
Xxx-Ala-Pro-Glu
   where Xxx is selected from the group consisting of Met, Ile, Val, Cys, Trp. Tyr, or Phe. This peptide motif is designated as CORE SEQUENCE (1-N). Peptides of four to twenty amino acid residues in length comprising CORE SEQUENCE (1-N) are designated as the peptides of CORE SEQUENCE (1-N). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (1), the peptides are selected from the group consisting of peptides of four to twenty amino acid residues in length, comprising the sequence:
Xxx-Ala-Pro-Glu
   where Xxx is selected from the group consisting of 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (1-U). Peptides of four to twenty amino acid residues in length comprising CORE SEQUENCE (1-U) are designated as the peptides of CORE SEQUENCE (1-U). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (1), the peptides are selected from the group consisting of peptides up to twenty amino acid residues in length comprising the sequence:
YMAPEV (SEQ ID NO:001), IIAPEG (SEQ ID NO:002), TWAPES (SEQ ID NO:016), AWAPEA (SEQ ID NO:024), PSAPEN (SEQ ID NO:026), HMAPEV (SEQ ID NO:027), YIAPEV (SEQ ID NO:028), KAPEPL (SEQ ID NO:029), WMAPET (SEQ ID NO:031), EAPEDL (SEQ ID NO:032), DVAPED (SEQ ID NO:033), YLAPEV (SEQ ID NO:034), YMAPEH (SEQ ID NO:035), WTAPEA (SEQ ID NO:037), WYAPEC (SEQ ID NO:038), YRAPEI (SEQ ID NO:040), and LIAPEA (SEQ ID NO:061). These peptides are designated as the peptides of GROUP (1-S).

In one embodiment of the peptides of CORE SEQUENCE (1), the peptides are selected from the group consisting of peptides up to twenty amino acid residues in length comprising the sequence:
YMAPEV (SEQ ID NO:001), IIAPEG (SEQ ID NO:002), TWAPES (SEQ ID NO:016), AWAPEA (SEQ ID NO:024), PSAPEN (SEQ ID NO:026), HMAPEV (SEQ ID NO:027), YIAPEV (SEQ ID NO:028), KAPEPL (SEQ ID NO:029), WMAPET (SEQ ID NO:031), EAPEDL (SEQ ID NO:032), DVAPED (SEQ ID NO:033), YLAPEV (SEQ ID NO:034), YMAPEH (SEQ ID NO:035), WTAPEA (SEQ ID NO:037), WYAPEC (SEQ ID NO:038), and YRAPEI (SEQ ID NO:040), with the proviso that peptides comprising the sequence LIAEAK (SEQ ID NO:009), peptides comprising the sequence LIANAK (SEQ ID NO:012), and peptides comprising SEQ ID NOS:041-113 are excluded. These peptides are designated as the peptides of GROUP (1-T).

In one embodiment of the peptides of CORE SEQUENCE (1), the peptides are selected from the group consisting of peptides of the sequence:
YMAPEV (SEQ ID NO:001), IIAPEG (SEQ ID NO:002), TWAPES (SEQ ID NO:016), AWAPEA (SEQ ID NO:024), PSAPEN (SEQ ID NO:026), HMAPEV (SEQ ID NO:027), YIAPEV (SEQ ID NO:028), KAPEPL (SEQ ID NO:029), WMAPET (SEQ ID NO:031), EAPEDL (SEQ ID NO:032), DVAPED (SEQ ID NO:033), YLAPEV (SEQ ID NO:034), YMAPEH (SEQ ID NO:035), WTAPEA (SEQ ID NO:037), WYAPEC (SEQ ID NO:038), YRAPEI (SEQ ID NO:040), and LIAPEA (SEQ ID NO:061). These peptides are designated as the peptides of GROUP (1-V).

In any of the foregoing or following embodiments, the peptides can be of length four to fifteen amino acids, four to twelve amino acids, four to ten amino acids, four to eight amino acids, six to twenty amino acids, six to fifteen amino acids, six to twelve amino acids, six to ten amino acids, or six to eight amino acids, provided that they contain the core sequence. Any of the foregoing embodiments can exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (1), the peptides are selected from the group consisting of peptides of the sequence:
YMAPEV (SEQ ID NO:001), IIAPEG (SEQ ID NO:002), TWAPES (SEQ ID NO:016), AWAPEA (SEQ ID NO:024), PSAPEN (SEQ ID NO:026), HMAPEV (SEQ ID NO:027), YIAPEV (SEQ ID NO:028), KAPEPL (SEQ ID NO:029), WMAPET (SEQ ID NO:031), EAPEDL (SEQ ID NO:032), DVAPED (SEQ ID NO:033), YLAPEV (SEQ ID NO:034), YMAPEH (SEQ ID NO:035), WTAPEA (SEQ ID NO:037), WYAPEC (SEQ ID NO:038), and YRAPEI (SEQ ID NO:040). These peptides are designated as the peptides of GROUP (1-W).

In one embodiment, the peptides are selected from the group consisting of peptides of six to twenty amino acid residues in length, comprising the sequence:
Xxx-Ala-Pro-D-Glu
   where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (2). Peptides of six to twenty amino acid residues in length comprising CORE SEQUENCE (2) are designated as the peptides of CORE SEQUENCE (2). In a further embodiment, the peptides of this motif exclude peptides comprising the sequence LIAP[D]EAK (SEQ ID NO:066), peptides comprising the sequence FIAP[D]EA (SEQ ID NO:069), and peptides comprising SEQ ID NOS:041-113.

In one embodiment of the peptides of CORE SEQUENCE (2), the peptides are selected from the group consisting of peptides of six to twenty amino acid residues in length, comprising the sequence:
Xxx-Ala-Pro-D-Glu
   where Xxx is selected from the group consisting of Met, Ile, Val, Cys, Trp, Tyr, or Phe. This peptide motif is designated as CORE SEQUENCE (2-N). Peptides of six to twenty amino acid residues in length comprising CORE SEQUENCE (2-N) are designated as the peptides of CORE SEQUENCE (2-N). In a further embodiment, the peptides of this motif exclude peptides comprising the sequence LIAP[D]EAK (SEQ ID NO:066), peptides comprising the sequence FIAP[D]EA (SEQ ID NO:069), and peptides comprising SEQ ID NOS:041-113.

In one embodiment of the peptides of CORE SEQUENCE (2), the peptides are selected from the group consisting of peptides of six to twenty amino acid residues in length, comprising the sequence:
Xxx-Ala-Pro-D-Glu
   where Xxx is selected from the group consisting of 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (2-U). Peptides of six to twenty amino acid residues in length comprising CORE SEQUENCE (2-U) are designated as the peptides of CORE SEQUENCE (2-U). In a further embodiment, the peptides of this motif exclude peptides comprising the sequence LIAP[D]EAK (SEQ ID NO:066), peptides comprising the sequence FIAP[D]EA (SEQ ID NO:069), and peptides comprising SEQ ID NOS:041-113.

In one embodiment of the peptides of CORE SEQUENCE (2), the peptides are selected from the group consisting of peptides up to twenty amino acid residues in length comprising the sequence: LFAP[D]EA (SEQ ID NO:013), LIAP[D]EA (SEQ ID NO:014), AWAP[D]EA (SEQ ID NO:025), or YMAP[D]EV (SEQ ID NO. 116). These peptides are designated as the peptides of GROUP (2-S).

In one embodiment of the peptides of CORE SEQUENCE (2), the peptides are selected from the group consisting of peptides up to twenty amino acid residues in length comprising the sequence: LFAP[D]EA (SEQ ID NO:013), LIAP[D]EA (SEQ ID NO:014), AWAP[D]EA (SEQ ID NO:025), or YMAP[D]EV (SEQ ID NO. 116), with the proviso that peptides comprising the sequence LIAEAK (SEQ ID NO:009), peptides comprising the sequence LIANAK (SEQ ID NO:012), and peptides comprising SEQ ID NOS:041-113 are excluded. These peptides are designated as the peptides of GROUP (2-T).

In any of the embodiments of the peptides disclosed herein, the peptides can be of length four to fifteen amino acids, four to twelve amino acids, four to ten amino acids, four to eight amino acids, six to twenty amino acids, six to fifteen amino acids, six to twelve amino acids, six to ten amino acids, or six to eight amino acids, provided that they contain the core sequence. Any of the disclosed embodiments of the peptides can exclude peptides comprising the sequence LIAP[D]EAK (SEQ ID NO:066), peptides comprising the sequence FIAP[D]EA (SEQ ID NO:069), and peptides comprising SEQ ID NOS:041-113.

In one embodiment of the peptides of CORE SEQUENCE (2), the peptides are selected from the group consisting of peptides of the sequence: LFAP[D]EA (SEQ ID NO:013), LIAP[D]EA (SEQ ID NO:014), AWAP[D]EA (SEQ ID NO:025), or YMAP[D]EV (SEQ ID NO. 116). These peptides are designated as the peptides of GROUP (2-V).

In one embodiment, the peptides are selected from the group consisting of peptides of four to twenty amino acid residues in length, comprising the sequence:
Xxx-Ala-Glu-Ala
   where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (3). In a further embodiment, the peptides of this motif exclude peptides comprising the sequence LIAEAK (SEQ ID NO:009). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113. Peptides of four to twenty amino acid residues in length comprising CORE SEQUENCE (3) are designated as the peptides of CORE SEQUENCE (3).

In one embodiment of the peptides of CORE SEQUENCE (3), the peptides are selected from the group consisting of peptides of four to twenty amino acid residues in length, comprising the sequence:
Xxx-Ala-Glu-Ala
   where Xxx is selected from the group consisting of Met, Ile, Val, Cys, Trp, Tyr, or Phe. This peptide motif is designated as CORE SEQUENCE (3-N). Peptides of four to twenty amino acid residues in length comprising CORE SEQUENCE (3-N) are designated as the peptides of CORE SEQUENCE (3-N). In a further embodiment, the peptides of CORE SEQUENCE (3-N) exclude peptides comprising the sequence LIAEAK (SEQ ID NO:009). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (3), the peptides are selected from the group consisting of peptides of four to twenty amino acid residues in length, comprising the sequence:
Xxx-Ala-Glu-Ala
   where Xxx is selected from the group consisting of 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (3-U). Peptides of four to twenty amino acid residues in length comprising CORE SEQUENCE (3-U) are designated as the peptides of CORE SEQUENCE (3-U). In a further embodiment, the peptides of CORE SEQUENCE (3-U) exclude peptides comprising the sequence LIAEAK (SEQ ID NO:009). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (3), the peptides are selected from the group consisting of peptides up to twenty amino acid residues in length having the sequence: EIAEAL (SEQ ID NO:003), SNVAEA (SEQ ID NO:004), ANIAEA (SEQ ID NO:005), LWAEAK (SEQ ID NO:008), LIAEAK (SEQ ID NO:009), LVAEAH (SEQ ID NO:011), Sar-Trp-Ala-Glu-Ala-NMeAl (SEQ ID NO:018), AWAEAK (SEQ ID NO:020), LVAEAK (SEQ ID NO:030), or SNVAEA (SEQ ID NO:039). These peptides are designated as the peptides of GROUP (3-S).

In another embodiment of the peptides of CORE SEQUENCE (3), the peptides are selected from the group consisting of peptides up to twenty amino acid residues in length comprising the sequence: EIAEAL (SEQ ID NO:003), SNVAEA (SEQ ID NO:004), ANIAEA (SEQ ID NO:005), LWAEAK (SEQ ID NO:008), LVAEAH (SEQ ID NO:011), Sar-Trp-Ala-Glu-Ala-NMeAl (SEQ ID NO:018), AWAEAK (SEQ ID NO:020), LVAEAK (SEQ ID NO:030), or SNVAEA (SEQ ID NO:039), with the proviso that peptides comprising the sequence LIAEAK (SEQ ID NO:009), peptides comprising the sequence LIANAK (SEQ ID NO:012), and peptides comprising SEQ ID NOS:041-113 are excluded. These peptides are designated as the peptides of GROUP (3-T).

In any of the foregoing embodiments, the peptides can be of length four to fifteen amino acids, four to twelve amino acids, four to ten amino acids, four to eight amino acids, six to twenty amino acids, six to fifteen amino acids, six to twelve amino acids, six to ten amino acids, or six to eight amino acids, provided that they contain the core sequence. Any of the foregoing embodiments can exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (3), the peptides are selected from the group consisting of peptides of the sequence: EIAEAL (SEQ ID NO:003), SNVAEA (SEQ ID NO:004), ANIAEA (SEQ ID NO:005), LWAEAK (SEQ ID NO:008), LIAEAK (SEQ ID NO:009), LVAEAH (SEQ ID NO:011), Sar-Trp-Ala-Glu-Ala-NMeAl (SEQ ID NO:018), AWAEAK (SEQ ID NO:020), LVAEAK (SEQ ID NO:030), or SNVAEA (SEQ ID NO:039). These peptides are designated as the peptides of GROUP (3-V).

In another embodiment of the peptides of CORE SEQUENCE (3), the peptides are selected from the group consisting of peptides of the sequence: EIAEAL (SEQ ID NO:003), SNVAEA (SEQ ID NO:004), ANIAEA (SEQ ID NO:005), LWAEAK (SEQ ID NO:008), LVAEAH (SEQ ID NO:011), Sar-Trp-Ala-Glu-Ala-NMeAl (SEQ ID NO:018), AWAEAK (SEQ ID NO:020), LVAEAK (SEQ ID NO:030), or SNVAEA (SEQ ID NO:039). These peptides are designated as the peptides of GROUP (3-W).

In one embodiment, the peptides are selected from the group consisting of peptides of four to twenty amino acid residues in length, comprising the sequence:
Xxx-Ala-D-Glu-Ala
   where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (4). Peptides of four to twenty amino acid residues in length comprising CORE SEQUENCE (4) are designated as the peptides of CORE SEQUENCE (4). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (4), the peptides are selected from the group consisting of peptides of four to twenty amino acid residues in length, comprising the sequence:
Xxx-Ala-D-Glu-Ala
   where Xxx is selected from the group consisting of Met, Ile, Val, Cys, Trp, Tyr, or Phe. This peptide motif is designated as CORE SEQUENCE (4-N). Peptides of four to twenty amino acid residues in length comprising CORE SEQUENCE (4-N) are designated as the peptides of CORE SEQUENCE (4-N). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (4), the peptides are selected from the group consisting of peptides of four to twenty amino acid residues in length, comprising the sequence:
Xxx-Ala-D-Glu-Ala
   where Xxx is selected from the group consisting of 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (4-U). Peptides of four to twenty amino acid residues in length comprising CORE SEQUENCE (4-U) are designated as the peptides of CORE SEQUENCE (4-U). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (4), the peptides are selected from the group consisting of peptides up to twenty amino acid residues in length comprising the sequence: Sar-Trp-Ala-[D]Glu-Ala-Sar (SEQ ID NO:019) or AWA[D]EAK (SEQ ID NO:021). These peptides are designated as the peptides of GROUP (4-S).

In one embodiment of the peptides of CORE SEQUENCE (4), the peptides are selected from the group consisting of peptides up to twenty amino acid residues in length comprising the sequence: Sar-Trp-Ala-[D]Glu-Ala-Sar (SEQ ID NO:019) or AWA[D]EAK (SEQ ID NO:021), with the proviso that peptides comprising the sequence LIAEAK (SEQ ID NO:009), peptides comprising the sequence LIANAK (SEQ ID NO:012), and peptides comprising SEQ ID NOS:041-113 are excluded. These peptides are designated as the peptides of GROUP (4-T).

In any of the foregoing embodiments, the peptides can be of length four to fifteen amino acids, four to twelve amino acids, four to ten amino acids, four to eight amino acids, six to twenty amino acids, six to fifteen amino acids, six to twelve amino acids, six to ten amino acids, or six to eight amino acids, provided that they contain the core sequence. Any of the foregoing embodiments can exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (4), the peptides are selected from the group consisting of peptides of the sequence: Sar-Trp-Ala-[D]Glu-Ala-Sar (SEQ ID NO:019) or AWA[D]EAK (SEQ ID NO:021). These peptides are designated as the peptides of GROUP (4-V).

In one embodiment, the peptides are selected from the group consisting of peptides of four to twenty amino acid residues in length, comprising the sequence:
Xxx-Ala-Asn-Ala
   where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (5). Peptides of four to twenty amino acid residues in length comprising CORE SEQUENCE (5) are designated as the peptides of CORE SEQUENCE (5). In a further embodiment, the peptides of CORE SEQUENCE (5) exclude peptides comprising the sequence LIANAK (SEQ ID NO:012). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (5), the peptides are selected from the group consisting of peptides of four to twenty amino acid residues in length, comprising the sequence:
Xxx-Ala-Asn-Ala
   where Xxx is selected from the group consisting of Met, Ile, Val, Cys, Trp, Tyr, or Phe. This peptide motif is designated as CORE SEQUENCE (5-N). Peptides of four to twenty amino acid residues in length comprising CORE SEQUENCE (5-N) are designated as the peptides of CORE SEQUENCE (5-N). In a further embodiment, the peptides of CORE SEQUENCE (5-N) exclude peptides comprising the sequence LIANAK (SEQ ID NO:012). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (5), the peptides are selected from the group consisting of peptides of four to twenty amino acid residues in length, comprising the sequence:
Xxx-Ala-Asn-Ala
   where Xxx is selected from the group consisting of 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (5-U). Peptides of four to twenty amino acid residues in length comprising CORE SEQUENCE (5-U) are designated as the peptides of CORE SEQUENCE (5-U). In a further embodiment, the peptides of CORE SEQUENCE (5-U) exclude peptides comprising the sequence LIANAK (SEQ ID NO:012). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (5), the peptides are selected from the group consisting of peptides up to twenty amino acid residues in length comprising the sequence: LIANAK (SEQ ID NO:012) or AWANAK (SEQ ID NO:022). These peptides are designated as the peptides of GROUP (5-S).

In one embodiment of the peptides of CORE SEQUENCE (5), the peptides are selected from the group consisting of peptides up to twenty amino acid residues in length comprising the sequence: AWANAK (SEQ ID NO:022), with the proviso that peptides comprising the sequence LIAEAK (SEQ ID NO:009), peptides comprising the sequence LIANAK (SEQ ID NO:012), and peptides comprising SEQ ID NOS:041-113 are excluded. These peptides are designated as the peptides of GROUP (5-T).

In any of the foregoing embodiments, the peptides can be of length four to fifteen amino acids, four to twelve amino acids, four to ten amino acids, four to eight amino acids, six to twenty amino acids, six to fifteen amino acids, six to twelve amino acids, six to ten amino acids, or six to eight amino acids, provided that they contain the core sequence. Any of the foregoing embodiments can exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (5), the peptides are selected from the group consisting of peptides of the sequence: LIANAK (SEQ ID NO:012) or AWANAK (SEQ ID NO:022). These peptides are designated as the peptides of GROUP (5-V).

In one embodiment of the peptides of CORE SEQUENCE (5), the peptides are selected from the group consisting of peptides of the sequence: AWANAK (SEQ ID NO:022). This peptide is designated as the peptide of GROUP (5-W).

In one embodiment, the peptides are selected from the group consisting of peptides of four to twenty amino acid residues in length, comprising the sequence:
Xxx-Ala-D-Asn-Ala
   where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (6). Peptides of four to twenty amino acid residues in length comprising CORE SEQUENCE (6) are designated as the peptides of CORE SEQUENCE (6). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (6), the peptides are selected from the group consisting of peptides of four to twenty amino acid residues in length, comprising the sequence:
Xxx-Ala-D-Asn-Ala
   where Xxx is selected from the group consisting of Met, Ile, Val, Cys, Trp, Tyr, or Phe. This peptide motif is designated as CORE SEQUENCE (6-N). Peptides of four to twenty amino acid residues in length comprising CORE SEQUENCE (6-N) are designated as the peptides of CORE SEQUENCE (6-N). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (6), the peptides are selected from the group consisting of peptides of four to twenty amino acid residues in length, comprising the sequence:
Xxx-Ala-D-Asn-Ala
   where Xxx is selected from the group consisting of 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (6-U). Peptides of four to twenty amino acid residues in length comprising CORE SEQUENCE (5) are designated as the peptides of CORE SEQUENCE (6-U). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (6), the peptides are selected from the group consisting of peptides up to twenty amino acid residues in length comprising the sequence: AWA[D]NAK (SEQ ID NO:023). These peptides are designated as the peptide of GROUP (6-S).

In one embodiment of the peptides of CORE SEQUENCE (6), the peptides are selected from the group consisting of peptides up to twenty amino acid residues in length comprising the sequence: AWA[D]NAK (SEQ ID NO:023), with the proviso that peptides comprising the sequence LIAEAK (SEQ ID NO:009), peptides comprising the sequence LIANAK (SEQ ID NO:012), and peptides comprising SEQ ID NOS:041-113 are excluded. These peptides are designated as the peptide of GROUP (6-T).

In any of the foregoing embodiments, the peptides can be of length four to fifteen amino acids, four to twelve amino acids, four to ten amino acids, four to eight amino acids, six to twenty amino acids, six to fifteen amino acids, six to twelve amino acids, six to ten amino acids, or six to eight amino acids, provided that they contain the core sequence. Any of the foregoing embodiments can exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (6), the peptides are selected from the group consisting of the peptide of the sequence: AWA[D]NAK (SEQ ID NO:023). This peptide is designated as the peptide of GROUP (6-V).

In one embodiment, the peptides are selected from the group consisting of peptides of four to twenty amino acid residues in length, comprising the sequence:
Xxx-Ala-Glu-Asn
   where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (7). Peptides of four to twenty amino acid residues in length comprising CORE SEQUENCE (7) are designated as the peptides of CORE SEQUENCE (7). In a further embodiment, the peptides of CORE SEQUENCE (7) exclude peptides comprising the sequence ANVAENA (SEQ ID NO:042). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (7), the peptides are selected from the group consisting of peptides of four to twenty amino acid residues in length, comprising the sequence:
Xxx-Ala-Glu-Asn
   where Xxx is selected from the group consisting of Met, Ile, Val, Cys, Trp, Tyr, or Phe. This peptide motif is designated as CORE SEQUENCE (7-N). Peptides of four to twenty amino acid residues in length comprising CORE SEQUENCE (7-N) are designated as the peptides of CORE SEQUENCE (7-N). In a further embodiment, the peptides of CORE SEQUENCE (7-N) exclude peptides comprising the sequence ANVAENA (SEQ ID NO:042). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (7), the peptides are selected from the group consisting of peptides of four to twenty amino acid residues in length, comprising the sequence:
Xxx-Ala-Glu-Asn
   where Xxx is selected from the group consisting of 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (7-U). Peptides of four to twenty amino acid residues in length comprising CORE SEQUENCE (7-U) are designated as the peptides of CORE SEQUENCE (7-U). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (7), the peptides are selected from the group consisting of peptides of four to twenty amino acid residues in length comprising the sequence: NVAENA (SEQ ID NO:010) or ANVAENA (SEQ ID NO:042). These peptides are designated as the peptides of GROUP (7-S).

In one embodiment of the peptides of CORE SEQUENCE (7), the peptides are selected from the group consisting of peptides of four to twenty amino acid residues in length comprising the sequence: NVAENA (SEQ ID NO:010), with the proviso that peptides comprising the sequence LIAEAK (SEQ ID NO:009), peptides comprising the sequence LIANAK (SEQ ID NO:012), and peptides comprising SEQ ID NOS:041-113 are excluded. These peptides are designated as the peptides of GROUP (7-T).

In any of the foregoing embodiments, the peptides can be of length four to fifteen amino acids, four to twelve amino acids, four to ten amino acids, four to eight amino acids, six to twenty amino acids, six to fifteen amino acids, six to twelve amino acids, six to ten amino acids, or six to eight amino acids, provided that they contain the core sequence. Any of the foregoing embodiments can exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (7), the peptides are selected from the group consisting of peptides of the sequence: NVAENA (SEQ ID NO:010) or ANVAENA (SEQ ID NO:042). These peptides are designated as the peptides of GROUP (7-V).

In one embodiment of the peptides of CORE SEQUENCE (7), the peptide is: NVAENA (SEQ ID NO:010). This peptide is designated as the peptide of GROUP (7-W).

In one embodiment, the peptides are selected from the group consisting of peptides of four to twenty amino acid residues in length, comprising the sequence:
Xxx-Ala-D-Glu-Asn
   where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (8). Peptides of four to twenty amino acid residues in length comprising CORE SEQUENCE (8) are designated as the peptides of CORE SEQUENCE (8). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (8), the peptides are selected from the group consisting of peptides of four to twenty amino acid residues in length, comprising the sequence:
Xxx-Ala-D-Glu-Asn
   where Xxx is selected from the group consisting of Met, Ile, Val, Cys, Trp, Tyr, or Phe. This peptide motif is designated as CORE SEQUENCE (8-N). Peptides of four to twenty amino acid residues in length comprising CORE SEQUENCE (8-N) are designated as the peptides of CORE SEQUENCE (8-N). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (8), the peptides are selected from the group consisting of peptides of four to twenty amino acid residues in length, comprising the sequence:
Xxx-Ala-D-Glu-Asn
   where Xxx is selected from the group consisting of 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala. This peptide motif is designated as CORE SEQUENCE (8-U). Peptides of four to twenty amino acid residues in length comprising CORE SEQUENCE (8) are designated as the peptides of CORE SEQUENCE (8-U). In a further embodiment, the peptides of this motif exclude peptides comprising the sequence LIAPEA (SEQ ID NO:061) and exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (8), the peptides are selected from the group consisting of peptides of four to twenty amino acid residues in length comprising the sequence: NVA[D]ENA (SEQ ID NO:114) or ANVA[D]ENA (SEQ ID NO:115). These peptides are designated as the peptides of GROUP (8-S).

In one embodiment of the peptides of CORE SEQUENCE (8), the peptides are selected from the group consisting of peptides of four to twenty amino acid residues in length comprising the sequence: NVA[D]ENA (SEQ ID NO:114) or ANVA[D]ENA (SEQ ID NO:115), with the proviso that peptides comprising the sequence LIAEAK (SEQ ID NO:009), peptides comprising the sequence LIANAK (SEQ ID NO:012), and peptides comprising SEQ ID NOS:041-113 are excluded. These peptides are designated as the peptides of GROUP (8-T).

In any of the foregoing embodiments, the peptides can be of length four to fifteen amino acids, four to twelve amino acids, four to ten amino acids, four to eight amino acids, six to twenty amino acids, six to fifteen amino acids, six to twelve amino acids, six to ten amino acids, or six to eight amino acids, provided that they contain the core sequence. Any of the foregoing embodiments can exclude peptides comprising the sequence LWAEAK (SEQ ID NO:008), peptides comprising the sequence LIAEAK (SEQ ID NO:009), peptides comprising the sequence LVAEAK (SEQ ID NO:030), peptides comprising the sequence LIANAK (SEQ ID NO:012), and peptides comprising SEQ ID NOS:041-113.

In one embodiment of the peptides of CORE SEQUENCE (8), the peptides are selected from the group consisting of peptides of the sequence: NVA[D]ENA (SEQ ID NO:114) or ANVA[D]ENA (SEQ ID NO:115). These peptides are designated as the peptides of GROUP (8-V).

In one embodiment, a combination comprising at least one peptide from CORE SEQUENCE (2) and at least one peptide from CORE SEQUENCE (7) is disclosed. In one embodiment, a combination comprising one peptide from CORE SEQUENCE (2) and one peptide from CORE SEQUENCE (7) is disclosed. In one embodiment, a combination of a peptide comprising the sequence YMAP[D]EV and a peptide comprising the sequence ANVAENA is disclosed. In one embodiment, a combination of the peptides YMAP[D]EV and ANVAENA is disclosed.

In a further embodiment, any set of peptides of CORE SEQUENCE (1), CORE SEQUENCE (1-N), CORE SEQUENCE (1-U), GROUP (1-S), GROUP (1-T), GROUP (1-V), GROUP (1-W), CORE SEQUENCE (2), CORE SEQUENCE (2-N), CORE SEQUENCE (2-U), GROUP (2-S), GROUP (2-T), GROUP (2-V), CORE SEQUENCE (3), CORE SEQUENCE (3-N), CORE SEQUENCE (3-U), GROUP (3-S), GROUP (3-T), GROUP (3-V), GROUP (3-W), CORE SEQUENCE (4), CORE SEQUENCE (4-N), CORE SEQUENCE (4-U), GROUP (4-S), GROUP (4-T), GROUP (4-V), CORE SEQUENCE (5), CORE SEQUENCE (5-N), CORE SEQUENCE (5-U), GROUP (5-S), GROUP (5-T), GROUP (5-V), GROUP (5-W), CORE SEQUENCE (6), CORE SEQUENCE (6-N), CORE SEQUENCE (6-U), GROUP (6-S), GROUP (6-T), GROUP (6-V), CORE SEQUENCE (7), CORE SEQUENCE (7-N), CORE SEQUENCE (7-U), GROUP (7-S), GROUP (7-T), GROUP (7-V), GROUP (7-W), CORE SEQUENCE (8), CORE SEQUENCE (8-N), CORE SEQUENCE (8-U), GROUP (8-S), GROUP (8-T), or GROUP (8-V), as recited in any part of this disclosure, can contain the additional proviso that peptides comprising the sequence LWAEAK (SEQ ID NO:008) are excluded: the additional proviso that peptides comprising the sequence LIAEAK (SEQ ID NO:009) are excluded: the additional proviso that peptides comprising the sequence LIANAK (SEQ ID NO:012) are excluded: the additional proviso that peptides comprising the sequence LVAEAK (SEQ ID NO:030) are excluded; the additional proviso that peptides comprising the sequence ANVAENA (SEQ ID NO:042) are excluded: the additional proviso that peptides comprising the sequence LIAEAK (SEQ ID NO:009) are excluded, the additional proviso that peptides comprising the sequence LIANAK (SEQ ID NO:012) are excluded, and peptides comprising the sequence ANVAENA (SEQ ID NO:042) are excluded: or the additional proviso that peptides comprising the sequence LWAEAK (SEQ ID NO:008), peptides comprising the sequence LIAEAK (SEQ ID NO:009), peptides comprising the sequence LIANAK (SEQ ID NO:012), peptides comprising the sequence LVAEAK (SEQ ID NO:030), and peptides comprising SEQ ID NOS:041-113 are excluded.

In a further embodiment, any set of peptides of CORE SEQUENCE (1), CORE SEQUENCE (1-N), CORE SEQUENCE (1-U), GROUP (1-S), GROUP (1-T), GROUP (1-V), GROUP (1-W), CORE SEQUENCE (2), CORE SEQUENCE (2-N), CORE SEQUENCE (2-U), GROUP (2-S), GROUP (2-T), GROUP (2-V), CORE SEQUENCE (3), CORE SEQUENCE (3-N), CORE SEQUENCE (3-U), GROUP (3-S), GROUP (3-T), GROUP (3-V), GROUP (3-W), CORE SEQUENCE (4), CORE SEQUENCE (4-N), CORE SEQUENCE (4-U), GROUP (4-S), GROUP (4-T), GROUP (4-V), CORE SEQUENCE (5), CORE SEQUENCE (5-N), CORE SEQUENCE (5-U), GROUP (5-S), GROUP (5-T), GROUP (5-V), GROUP (5-W), CORE SEQUENCE (6), CORE SEQUENCE (6-N), CORE SEQUENCE (6-U), GROUP (6-S), GROUP (6-T), GROUP (6-V), CORE SEQUENCE (7), CORE SEQUENCE (7-N), CORE SEQUENCE (7-U), GROUP (7-S), GROUP (7-T), GROUP (7-V), GROUP (7-W), CORE SEQUENCE (8), CORE SEQUENCE (8-N), CORE SEQUENCE (8-U), GROUP (8-S), GROUP (8-T), or GROUP (8-V), as recited in any part of this disclosure, can contain the additional proviso that one or more peptides comprising the sequence of any one or more of SEQ ID NOS:001-113 in any combination are excluded, with the further proviso that at least one peptide sequence remains in the resulting set.

In the sequences described above and any other sequence disclosed herein, methionine (Met) may be replaced with norleucine (Nor).

In any of the embodiments of the specific peptide sequences disclosed herein, and the methods of use of the peptide sequences disclosed herein, homologous sequences having at least about 70%, at least about 75%, at least about 80%, at least about 85%, at least about 90%, or at least about 95% sequence homology can be used, provided that the core sequence is maintained.

In any of the embodiments of the specific peptide sequences disclosed herein, and the methods of use of the peptide sequences disclosed herein, substitutions can be made in the non-core regions of the sequence by replacing one, two, or three amino acids with a homologous amino acid. Thus, negatively charged amino acids can be substituted for other negatively charged amino acids (Asp, Glu); positively charged amino acids can be substituted for other positively charged amino acids (Lys, Arg. His); hydrophobic amino acids can be substituted for other hydrophobic amino acids (Ala, Val, Ile, Leu, Met); hydrophilic amino acids can be substituted for other hydrophilic amino acids (Ser, Thr) (Asn, Gln); aromatic amino acids can be substituted for other aromatic amino acids (Phe, Tyr, Trp, His); and less bulky amino acids can be substituted for other less bulky amino acids (Ala, Gly). The groups of amino acids in parentheses indicate amino acids considered homologous for purposes of substitution.

In one embodiment, disclosed herein is a method of treating COVID-19 in a patient in need thereof, comprising administering a composition comprising a therapeutically effective amount of a compound which inhibits NF κB activation or inhibits NF κB activity. In a further embodiment, the composition comprises a therapeutically effective amount of one or more peptides selected from the group consisting of peptides of CORE SEQUENCE (1), CORE SEQUENCE (1-N), CORE SEQUENCE (1-U), GROUP (1-S), GROUP (1-T), GROUP (1-V), GROUP (1-W), CORE SEQUENCE (2), CORE SEQUENCE (2-N), CORE SEQUENCE (2-U), GROUP (2-S), GROUP (2-T), GROUP (2-V), CORE SEQUENCE (3), CORE SEQUENCE (3-N), CORE SEQUENCE (3-U), GROUP (3-S), GROUP (3-T), GROUP (3-V), GROUP (3-W), CORE SEQUENCE (4), CORE SEQUENCE (4-N), CORE SEQUENCE (4-U), GROUP (4-S), GROUP (4-T), GROUP (4-V), CORE SEQUENCE (5), CORE SEQUENCE (5-N), CORE SEQUENCE (5-U), GROUP (5-S), GROUP (5-T), GROUP (5-V), GROUP (5-W), CORE SEQUENCE (6), CORE SEQUENCE (6-N), CORE SEQUENCE (6-U), GROUP (6-S), GROUP (6-T), GROUP (6-V), CORE SEQUENCE (7), CORE SEQUENCE (7-N), CORE SEQUENCE (7-U), GROUP (7-S), GROUP (7-T), GROUP (7-V), GROUP (7-W), CORE SEQUENCE (8), CORE SEQUENCE (8-N), CORE SEQUENCE (8-U), GROUP (8-S), GROUP (8-T), GROUP (8-V), SEQ ID NOS:001-069, (SEQ ID NOS:001-007, 010, 011, 013-029, and 031-040), and (SEQ ID NOS:001-007, 010, 011, 013-029, and 031-040) to the patient. In one embodiment, the composition additionally comprises a pharmaceutically acceptable carrier. In one embodiment, the administration is intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (1) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (1-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (1-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (1-S) injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (1-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (2) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (2-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (2-U) to the patient. In one embodiment, the composition additionally comprises a pharmaceutically acceptable carrier. In one embodiment, the administration is via intravenous injection. In one embodiment, the administration is via inhalation or pulmonary administration.

In need thereof, comprising administering a composition comprising a therapeutically effective amount of one or more peptides selected from the group consisting of GROUP (2-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (2-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (2-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (3) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (3-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (3-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (3-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (3-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (3-V) to the patient. In one embodiment, the composition additionally comprises a pharmaceutically acceptable carrier. In one embodiment, the administration is via intravenous injection. In one embodiment, the administration is via inhalation or pulmonary administration.

In need thereof, comprising administering a composition comprising a therapeutically effective amount of one or more peptides selected from the group consisting of GROUP (3-W) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (4) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (4-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (4-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (4-S) injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (4-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (4-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (5) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (5-N) to the patient. In one embodiment, the composition additionally comprises a pharmaceutically acceptable carrier. In one embodiment, the administration is via intravenous injection. In one embodiment, the administration is via inhalation or pulmonary administration.

In need thereof, comprising administering a composition comprising a therapeutically effective amount of one or more peptides selected from the group consisting of CORE SEQUENCE (5-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (5-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (5-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (5-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (5-W) to the patient. In one embodiment, the composition additionally comprises a injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (6) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (6-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (6-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (6-S) to the patient. In one embodiment, the composition additionally comprises a pharmaceutically acceptable carrier. In one embodiment, the administration is via intravenous injection. In one embodiment, the administration is via inhalation or pulmonary administration.

In need thereof, comprising administering a composition comprising a therapeutically effective amount of one or more peptides selected from the group consisting of GROUP (6-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (6-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (7) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (7-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (7-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (7-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (7-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (7-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (7-W) to the patient. In one embodiment, the composition additionally comprises a pharmaceutically acceptable carrier. In one embodiment, the administration is via intravenous injection. In one embodiment, the administration is via inhalation or pulmonary administration.

In need thereof, comprising administering a composition comprising a therapeutically effective amount of one or more peptides selected from the group consisting of CORE SEQUENCE (8) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (8-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of CORE SEQUENCE (8-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (8-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (8-T) injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of GROUP (8-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of SEQ ID NOS:001 to 040 to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of SEQ ID NOS:001-008, 010, 011, and 013-040 to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of one or more peptides selected from the group consisting of SEQ ID NOS:041-069 to the patient. In one embodiment, the composition additionally comprises a pharmaceutically acceptable carrier. In one embodiment, the administration is via intravenous injection. In one embodiment, the administration is via inhalation or pulmonary administration.

The peptides and treatments as described herein are particularly useful for treatment of COVID-19, but are useful for treatment of any disease or disorder that results in perturbation of the immune system leading to inflammation. For example, the peptides and treatments can be use in patients infected by another COVID variant, or another virus. The peptides and treatments can be used for COVID, seasonal flu, pneumonia, chronic obstructive pulmonary disorder, tuberculosis, or any disease that leads to perturbation in the immune system resulting in inflammation.

In one embodiment, disclosed herein is a method of treating pneumonia in a patient in need thereof, comprising administering a composition comprising a therapeutically effective amount of a compound which inhibits NF κB activation or inhibits NF κB activity. In a further embodiment, the composition comprises a therapeutically effective amount of one or more peptides selected from the group consisting of peptides of CORE SEQUENCE (1), CORE SEQUENCE (1-N), CORE SEQUENCE (1-U), GROUP (1-S), GROUP (1-T), GROUP (1-V), GROUP (1-W), CORE SEQUENCE (2), CORE SEQUENCE (2-N), CORE SEQUENCE (2-U), GROUP (2-S), GROUP (2-T), GROUP (2-V), CORE SEQUENCE (3), CORE SEQUENCE (3-N), CORE SEQUENCE (3-U), GROUP (3-S), GROUP (3-T), GROUP (3-V), GROUP (3-W), CORE SEQUENCE (4), CORE SEQUENCE (4-N), CORE SEQUENCE (4-U), GROUP (4-S), GROUP (4-T), GROUP (4-V), CORE SEQUENCE (5), CORE SEQUENCE (5-N), CORE SEQUENCE (5-U), GROUP (5-S), GROUP (5-T), GROUP (5-V), GROUP (5-W), CORE SEQUENCE (6), CORE SEQUENCE (6-N), CORE SEQUENCE (6-U), GROUP (6-S), GROUP (6-T), GROUP (6-V), CORE SEQUENCE (7), CORE SEQUENCE (7-N), CORE SEQUENCE (7-U), GROUP (7-S), GROUP (7-T), GROUP (7-V), GROUP (7-W), CORE SEQUENCE (8), CORE SEQUENCE (8-N), CORE SEQUENCE (8-U), GROUP (8-S), GROUP (8-T), GROUP (8-V), SEQ ID NOS:001-069, (SEQ ID NOS:001-007, 010, 011, 013-029, and 031-040), and (SEQ ID NOS:001-007, 010, 011, 013-029, and 031-040) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (1) to the patient. In one embodiment, the composition additionally comprises a injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (1-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (1-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (1-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (1-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (2) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (2-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (2-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (2-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (2-T) injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (2-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (3) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (3-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (3-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (3-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (3-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (3-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (3-W) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (4) to the patient. In one embodiment, the composition additionally comprises a injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (4-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (4-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (4-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (4-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (4-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (5) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (5-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (5-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (5-S) injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (5-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (5-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (5-W) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (6) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (6-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (6-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (6-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (6-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (6-V) to the patient. In one embodiment, the composition additionally comprises a injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (7) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (7-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (7-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (7-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (7-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (7-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (7-W) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (8) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (8-N) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of CORE SEQUENCE (8-U) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (8-S) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (8-T) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of GROUP (8-V) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of SEQ ID NOS:001 to 040 to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of SEQ ID NOS:001-008, 010, 011, and 013-040 to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating pneumonia in a patient in need thereof, comprising administering a composition comprising a therapeutically effective amount of one or more peptides selected from the group consisting of SEQ ID NOS:041-069 to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of a combination comprising at least one peptide from CORE SEQUENCE (2) and at least one peptide from CORE SEQUENCE (7). In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of a combination comprising a peptide from CORE SEQUENCE (2) and a peptide from CORE SEQUENCE (7). In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of a combination comprising a peptide comprising the sequence YMAP[D]EV and a peptide comprising the sequence ANVAENA. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID-19 in a patient in need thereof, comprising administering a composition comprising a therapeutically effective amount of a combination comprising the peptide YMAP[D]EV and the peptide ANVAENA. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein are methods of treating COVID-19 in a patient in need thereof, comprising administering to the patient an amount of one or more peptides disclosed herein, said amount effective to reduce expression of a gene, where expression of said gene promotes inflammation or contributes to the activity of NF kappa B.

In one embodiment, disclosed herein is a method of treating COVID variants, influenza, chronic obstructive pulmonary disorder, or tuberculosis in a patient in need thereof, comprising administering a composition which comprises a therapeutically effective amount of one or more peptides selected from the group consisting of peptides of CORE SEQUENCE (1), CORE SEQUENCE (1-N), CORE SEQUENCE (1-U), GROUP (1-S), GROUP (1-T), GROUP (1-V), GROUP (1-W), CORE SEQUENCE (2), CORE SEQUENCE (2-N), CORE SEQUENCE (2-U), GROUP (2-S), GROUP (2-T), GROUP (2-V), CORE SEQUENCE (3), CORE SEQUENCE (3-N), CORE SEQUENCE (3-U), GROUP (3-S), GROUP (3-T), GROUP (3-V), GROUP (3-W), CORE SEQUENCE (4), CORE SEQUENCE (4-N), CORE SEQUENCE (4-U), GROUP (4-S), GROUP (4-T), GROUP (4-V), CORE SEQUENCE (5), CORE SEQUENCE (5-N), CORE SEQUENCE (5-U), GROUP (5-S), GROUP (5-T), GROUP (5-V), GROUP (5-W), CORE SEQUENCE (6), CORE SEQUENCE (6-N), CORE SEQUENCE (6-U), GROUP (6-S), GROUP (6-T), GROUP (6-V), CORE SEQUENCE (7), CORE SEQUENCE (7-N), CORE SEQUENCE (7-U), GROUP (7-S), GROUP (7-T), GROUP (7-V), GROUP (7-W), CORE SEQUENCE (8), CORE SEQUENCE (8-N), CORE SEQUENCE (8-U), GROUP (8-S), GROUP (8-T), GROUP (8-V), SEQ ID NOS:001-069, (SEQ ID NOS:001-007, 010, 011, 013-029, and 031-040), and (SEQ ID NOS:001-007, 010, 011, 013-029, and 031-040) to the patient. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID variants, influenza, chronic obstructive pulmonary disorder, or tuberculosis in a patient in need thereof, comprising administering a composition comprising a therapeutically effective amount of a combination comprising a peptide from CORE SEQUENCE (2) and a peptide from CORE SEQUENCE (7). In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID variants, influenza, chronic obstructive pulmonary disorder, or tuberculosis in a patient in need thereof, comprising administering a composition comprising a therapeutically effective amount of a combination comprising a peptide comprising the sequence YMAP[D]EV and a peptide comprising the sequence ANVAENA. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID variants, influenza, chronic obstructive pulmonary disorder, or tuberculosis in a patient in need thereof, comprising administering a composition comprising a therapeutically effective amount of a peptide comprising the sequence YMAP[D]EV. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein is a method of treating COVID variants, influenza, chronic obstructive pulmonary disorder, or tuberculosis in a patient in need thereof, comprising administering a composition comprising a therapeutically effective amount of a combination comprising the peptide YMAP[D]EV and the peptide ANVAENA. In one embodiment, the composition additionally comprises a pharmaceutically acceptable carrier. In one embodiment, the administration is via intravenous injection. In one embodiment, the administration is via inhalation or pulmonary administration.

In one embodiment, disclosed herein are methods of treating COVID variants, influenza, chronic obstructive pulmonary disorder, or tuberculosis in a patient in need thereof, comprising administering to the patient an amount of one or more peptides disclosed herein, said amount effective to reduce expression of a gene, where expression of said gene promotes inflammation or contributes to the activity of NF kappa B.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition is described as “consisting essentially of” the listed components, the composition contains the components expressly listed, and may contain other components which do not substantially affect the condition being treated such as inert excipients or carriers. However, the composition either does not contain any other components which do substantially affect the condition being treated other than those components expressly listed: or, if the composition does contain extra components other than those listed which substantially affect the condition being treated, the composition does not contain a sufficient concentration or amount of those extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed.

The compositions and methods described herein, including any embodiment described herein, may be used alone or may be used in combination with other compositions and methods.

### BRIEF DESCRIPTION OF THE DRAWINGS

FIG. 1 shows the pathway by which infection with COVID-19 causes lung and systemic pathology.

FIG. 2A, FIG. 2B, and FIG. 2C show peptides for use in the methods disclosed herein. FIG. 2A shows SEQ IDS NOS:001 through 040. SEQ IDS NOS:009 and 012 are peptides disclosed in U.S. Pat. Nos. 5,661,127, 5,780,436; and 6,638,912. FIG. 2B shows SEQ IDS NOS:041 through 069, which are peptides disclosed in U.S. Pat. Nos. 5,661,127, 5,780,436; and 6,638,912. FIG. 2C shows SEQ ID NOS:070 through 113, which are peptides disclosed in U.S. Patent Application Publication No. 2006/0293228, and also shows SEQ ID NOS:114 through 116.

FIG. 3A lists the naturally-occurring (proteinogenic) encoded amino acids. FIG. 3B lists unnatural and/or non-encoded (non-proteinogenic) amino acids.

FIG. 4 shows results of treatment with YMAP[D]EV peptide or control in a paw inflammation assay. Upper curve, control animals: lower curve, treated with peptide.

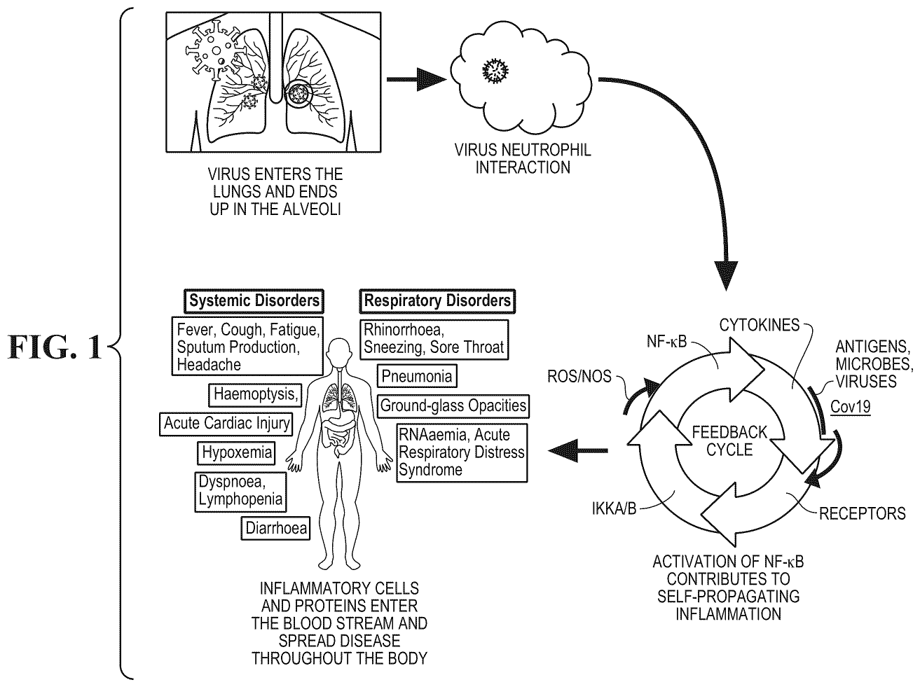

### DETAILED DESCRIPTION

The current disclosure provides compositions for use in treating lung disorders, including pneumonia. The lung disorders can be caused by viral or bacterial infection. The viral infection can be the disease caused by SARS-COV-2, that is, the disease COVID-19. The current disclosure also provides compositions for use in treating inflammatory diseases and disorders.

### Definitions

The following definitions are used herein.

A “subject,” “individual,” or “patient” is a vertebrate, preferably a mammal, more preferably a human. In other embodiments, the subject, individual, or patient is a food animal, such as a chicken, turkey, duck, goose, cow, lamb, sheep, pig, or goat. In other embodiments, the subject, individual, or patient is a domestic animal, such as a cat, dog, bird, rabbit, or guinea pig. The compounds, compositions, and methods disclosed herein can be used in human medicine and in veterinary medicine.

“Treating” a disease or disorder with the compounds and methods discussed herein is defined as administering one or more of the compounds discussed herein, with or without additional therapeutic agents, in order to reduce or eliminate either the disease or disorder or one or more symptoms of the disease or disorder, or to retard the progression of the disease or disorder or of one or more symptoms of the disease or disorder, or to reduce the severity of the disease or disorder or of one or more symptoms of the disease or disorder. “Suppression” of a disease or disorder with the compounds and methods discussed herein is defined as administering one or more of the compounds discussed herein, with or without additional therapeutic agents, in order to suppress the clinical manifestation of the disease or disorder, or to suppress the manifestation of adverse symptoms of the disease or disorder. The distinction between treatment and suppression is that treatment occurs after adverse symptoms of the disease or disorder are manifest in a subject, while suppression occurs before adverse symptoms of the disease or disorder are manifest in a subject. Suppression may be partial, substantially total, or total.

“Therapeutic use” of the compounds discussed herein is defined as using one or more of the compounds discussed herein to treat or suppress a disease or disorder, as defined above. A “therapeutically effective amount” of a compound is an amount of the compound, which, when administered to a subject, is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder, or to suppress the clinical manifestation of a disease or disorder, or to suppress the manifestation of adverse symptoms of a disease or disorder. A therapeutically effective amount can be given in one or more administrations.

As used herein, the singular forms “a,” “an,” “or,” and “the” include plural referents unless the context clearly dictates otherwise.

Reference to “about” a value or parameter herein includes and describes variations that are directed to that value or parameter itself, as well as other values encompassed by “about.” For example, description referring to “about X” includes description of the value “X.”

The symbol [D] before an amino acid abbreviation indicates the D-isomer of the amino acid immediately following the symbol. For example, in the peptide YMAP[D]EV, the glutamic acid residue [D]E is D-glutamic acid, while the remaining amino acids are L-amino acids, and the peptide is L-tyrosyl-L-methionyl-L-alanyl-L-prolyl-D-glutamyl-L-valine.

“Administration near the affected area” indicates that a compound which inhibits NF κB activity is administered in sufficient proximity to a site of pathology, such that the compound is able to exert a therapeutic effect on the pathology.

“Inhibitors of NF κB,” “inhibition of NF κB,” and “NF κB inhibitors” refers to compounds which inhibit the activation or activity of NF κB by any mechanism. The terms “inhibitors of NF κB,” “inhibition of NF κB,” and “NF κB inhibitors” do not require or necessarily imply direct binding to NF κB; such binding may or may not occur when NF κB activity is inhibited.

“Peptoids” refer to peptide-like polymers comprising poly-N-substituted glycines. In peptoids, amino acid monomers are replaced with monomers where the side chain formerly attached to the alpha carbon of the amino acid is attached to the amino group instead, and the side chain on the alpha carbon has been replaced with hydrogen. Peptoids that can be used in the methods described herein have the same sequence as the peptides indicated here, where the residues in the peptides have been replaced by their peptoid equivalents (for example, alanine in a peptide is replaced with N-methyl glycine). Where a residue indicated in a sequence already bears a non-hydrogen amino substituent, such as proline, that reside remains unchanged when occurring in a peptoid.

“Peptide mimetics” refer to peptides where one or more of the peptide bonds are replaced with an ester bond (—(C═O)—O— instead of —(C═O)—NH—; depsipeptides), where one or more of the peptide bonds are replaced with a thioester bond (—(C═O)—S— instead of —(C═O)—NH—), or where the carbonyl of one or more of the peptide bonds are replaced with a methylene group (—(CH₂)—NH— instead of —(C═O)—NH—; reduced amide bonds). Methods for making depsipeptides are described in Stawikowski et al, Methods Mol. Biol. 386:321-39 (2007) and Albericio et al., Org. Lett. 7(4):597-600 (2005). Methods for making peptides with thioester bonds are described in Raz et al., Org. Lett. 13(7):1606-1609 (2011) and Stuhr-Hansen et al., European Journal of Organic Chemistry 2013(24):5290-5294 (2013). Methods for making peptides with reduced amide bonds are described in Sasaki et al., Peptides 8(1): 119-121 and Meyer et al., J. Med. Chem. 38(18):3462-3468 (1995).

While the peptide compounds described herein can occur and can be used as the neutral (non-salt) compounds (including zwitterionic non-salt compounds), the description is intended to embrace all salts of the compounds described herein, as well as methods of using such salts of the compounds. In one embodiment, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N-dibenzylethylenediamine, and triethylamine salts. Salts of acidic compounds with amino acids, such as lysine salts, can also be prepared.

The compounds can be administered in prodrug form. Prodrugs are derivatives of the compounds, which are themselves relatively inactive but which convert into the active compound when introduced into the subject in which they are used by a chemical or biological process in vivo, such as an enzymatic conversion. Suitable prodrug formulations include, but are not limited to, peptide conjugates of the compounds disclosed herein and esters of compounds disclosed herein. Further discussion of suitable prodrugs is provided in H. Bundgaard, Design of Prodrugs, New York: Elsevier, 1985; in R. Silverman, The Organic Chemistry of Drug Design and Drug Action, Boston: Elsevier, 2004: in R. L. Juliano (ed.), Biological Approaches to the Controlled Delivery of Drugs (Annals of the New York Academy of Sciences, v. 507), New York: New York Academy of Sciences, 1987; and in E. B. Roche (ed.), Design of Biopharmaceutical Properties Through Prodrugs and Analogs (Symposium sponsored by Medicinal Chemistry Section, APhA Academy of Pharmaceutical Sciences, November 1976 national meeting, Orlando, Florida), Washington: The Academy, 1977.

DESCRIPTION OF COMPOUNDS DISCLOSED HEREIN: The compounds as described herein are 4-20 residue peptides, peptide-mimetics, or peptoids. When necessary for clarity, the term “standard peptide” is used to refer to polymers of alpha-amino acids and/or imino acids (for example, proline) without peptide bond modification. The compounds have specific tri-peptide core sequences within the body of the peptide as described below. The peptides are designed to emulate a common conformational feature present in protein kinases, both serine threonine kinases and protein tyrosine kinases. These peptides can assume conformations simulating certain interactive domains in signaling protein kinases, and they can modulate the activities of kinases as molecular decoys and by allosteric mechanisms.

CHARACTERISTICS OF PEPTIDES DISCLOSED HEREIN: The disclosed peptides mimic important physicochemical characteristics of certain interactive structural features in serine threonine kinases. These features can be simulated by many amino acid sequences. The activity of these peptides is dependent on the presence of one of several contiguous sequences within the body of the peptide. This sequence will be referred to as the “core sequence” hereinafter. The core sequences, each made up of four residues emulate surfaces presented by certain interactive sites present in numerous protein kinases, both protein tyrosine kinases and in serine threonine kinases. While none of these domains may participate in the catalytic activity of the enzymes, their presence both in the enzymes and in the substrate protein kinases is essential for intermolecular recognition and interaction. The peptide design is based on the argument that interactive sites on proteins need to be near the surface, that they may exist in more than one isoenergic conformation one of which presents an optimal surface to couple with a complementary receiving site on the interacting partner, that the interaction is fully consummated by induced fit, that the interactions typically involve weak bonds, and finally, the interactive domains do not need to be large. In silico interaction studies between several of the disclosed peptides and a number of protein kinases, and additional studies, confirm stable peptide-kinase interactions. Specifically, these studies predict interaction with the similar receiving site on different kinases. Thus, it is postulate that the peptides alter kinase activities by mimicking specific molecular domains on one partner (say Protein A) in its interaction with its specific receiving site on Protein B. Thus the peptides may be referred to as molecular decoys. The core sequences essential for the activity of the peptides are described below. While many of the amino acid residues listed here do not occur naturally in animal proteins, they are included here for their ability to contribute to peptide surfaces mimicking domains found in numerous enzymes and proteins associated with the activation of NF κB.

The peptides as disclosed herein contain various core sequences found to confer NF κB inhibitory activity. The peptides as disclosed herein act to restore normal homeostasis of NF kappa B, and affect mitotic and immune system checkpoints to prevent cellular inflammation. The peptides can range in length from four to twenty amino acids, four to fifteen amino acids, four to twelve amino acids, four to ten amino acids, four to eight amino acids, six to twenty amino acids, six to fifteen amino acids, six to twelve amino acids, six to ten amino acids, or six to eight amino acids, as long as the peptide comprises the appropriate core sequence. For peptides indicated as comprising a specific sequence, the lower limit of the peptide length is the specific sequence indicated, while the upper limit of the peptide length can be twenty amino acids, fifteen amino acids, twelve amino acids, ten amino acids, or eight amino acids, with the proviso that the upper limit of peptide length is greater than or equal to the lower limit of peptide length (that is, the upper limit of the peptide length cannot be shorter than the sequence specified for the peptide).

Where a peptide is described as having a certain length comprising a specific sequence, the specific sequence indicated is contained within the length of the peptide, without any insertions or alterations to the sequence. For example, a peptide of length ten amino acids comprising the sequence ANVAENA can have additional amino acids preceding ANVAENA in the overall sequence, or additional amino acids following ANVAENA in the overall sequence, but insertions of other amino acids in ANVAENA are not permitted.

In the sequences described above and any other sequence disclosed herein, Met may be replaced with the isostere norleucine (Nor).

In some embodiments, the residues on the C-terminus of the core sequences described above and any other sequence disclosed herein, may be un-natural amino acids such as 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, or β-tert-butyl-Ala.

In some embodiments the residue on the C-terminus of the core sequences described above and any other sequence disclosed herein, may be un-natural amino acids such as 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid. β-cyclohexyl-Ala, dehydroalanine, or β-tert-butyl-Ala.

In other embodiments the residues on both the N- and C-terminus of the core sequences described above, and any other sequence disclosed herein, may be un-natural amino acids such as 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, or β-tert-butyl-Ala.

In some embodiments, the residues on the N- and C-termini of the core peptide sequences described above, and any other sequence disclosed herein, may be residues or compounds designed to protect the peptide from degradation by proteases or peptidases.

In another embodiment the peptide active core may be extended with additional amino acids on its N-terminus, or on its C-terminus, or on both termini such that the total number of amino acids may not exceed twenty.

In other embodiments, the active core sequence as described above, and any other sequence disclosed herein, or its extended forms as described herein may be coupled to a larger molecule such as polyethylene glycol, or a polysaccharide, to enhance its dwell time in tissues and in organs including the alimentary canal.

Peptides in various embodiments as described above may be modified to produce pro-drugs that would be transformed to the active form in physiologic milieux.

Peptides in various embodiments as described above may be coupled to a lipid molecule, to enhance penetration in tissues and dwell time in tissues and organs including the alimentary canal.

Peptides in various embodiments as described above may be modified to increase their stability in the presence of proteases present in tissue fluids, in serum, saliva, in the alimentary canal, and in various body fluids in the tissues under treatment. Such protective modifications include, but are not restricted to:
Addition of a D-amino acid on the N-terminus of the peptide;
Addition of a D-amino acid on the C-terminus of the peptide;
Acetylation of the N-terminal amino group;
Modification of the N-terminal amino group with 1-amino-cyclohexane-carboxylic acid;
Modification of the N-terminal amino group with β-acetyl-2,3-diamino propionic acid;
Amidation of the C-terminal carboxyl group with —NH₂; and
Esterification of the C-terminal carboxyl group with —OCH₃ or —OCH₂CH₃.

U.S. Pat. No. 10,030,048, International Patent Application No. WO 2016/049580, U.S. Pat. Nos. 5,661,127, 5,780,436, and 6,638,912, and U.S. Patent Publication No. 2006/0293228 are incorporated herein by reference in their entireties, and in particular, the lists of peptides therein are incorporated herein by reference.

In some embodiments, the total length of the peptide including core sequences may be seven, eight, nine, ten, eleven, twelve, thirteen, fourteen, fifteen, sixteen, seventeen, eighteen, nineteen or twenty residues, such that in a peptide nX(CORE)mX, where n is the number of residues X on the N-terminal of the core sequence, and m is the number of residues X on the C-terminal of the core sequence, the sum of n+m+the number of residues in the CORE sequence is five, six, seven, eight, nine, ten, eleven, twelve, thirteen, fourteen, fifteen, sixteen, seventeen, eighteen, nineteen or twenty.

In other embodiments, the numbers n and m may range from zero to sixteen, such that the core sequence may be part of a longer sequence in which the residues X on the N-terminal side and on the C-terminal side of the CORE sequence may not be the same, thus, the following sequences are contemplated:
(CORE)-mX where there may be zero to sixteen residues on the C-terminal side of the core sequence, i.e., m is the number of residues X on the C-terminus of the peptide, and each residue X is chosen independently of the other residues;
nX-(CORE) where there may be zero to sixteen residues on the N-terminal side of the core sequence, i.e., n refers to the number of residues on the N-terminal side of the core sequence, and each residue X is chosen independently of the other residues; and
nX-(CORE)-mX where the numbers n and m are independently variable, and add up to zero to sixteen, and each residue X is chosen independently of the other residues.

In any of the embodiments of the specific peptide sequences disclosed herein, homologous sequences having at least about 70%, at least about 75%, at least about 80%, at least about 85%, at least about 90%, or at least about 95% sequence homology can be used, provided that the core sequence is maintained.

In any of the embodiments of the specific peptide sequences disclosed herein, substitutions can be made in the non-core regions of the sequence by replacing one, two, or three amino acids with a homologous amino acid. Thus, negatively charged amino acids can be substituted for other negatively charged amino acids (Asp, Glu); positively charged amino acids can be substituted for other positively charged amino acids (Lys, Arg, His); hydrophobic amino acids can be substituted for other hydrophobic amino acids (Ala, Val, Ile, Leu, Met); hydrophilic amino acids can be substituted for other hydrophilic amino acids (Ser, Thr) (Asn, Gln); aromatic amino acids can be substituted for other aromatic amino acids (Phe, Tyr, Trp, His); and less bulky amino acids can be substituted for other less bulky amino acids (Ala, Gly). The groups of amino acids in parentheses indicate amino acids considered homologous for purposes of substitution.

SYNTHESIS OF PEPTIDES: The synthesis of peptides is commonly practiced and a person familiar with the art can easily reproduce the compounds disclosed here. Most commonly, peptides are synthesized in commercially available instruments, each of which is provided with directions and methods. Peptides may also be obtained from a large number of companies that specialize in the manufacture of peptides. The peptides disclosed herein may be synthesized by any suitable method for producing peptides of a given sequence. Peptides can be synthesized by solid phase synthesis, manual or automated, as first developed by Merrifield and described by Stewart et al. in Solid Phase Peptide Synthesis (1984), by Bodansky in Peptide Synthesis (1976) and, Stewart et al. in Solid Phase Peptide Synthesis (1984), or W. C. Chan and Peter D. White (Editors), Fmoc Solid Phase Peptide Synthesis: A Practical Approach (The Practical Approach Series) 1st Edition, Oxford: Oxford University Press, 2000.

After synthesis, peptides can be purified by high-performance liquid chromatography or by other methods known in the art. The peptides can be purified to about 70%, about 80%, about 90%, about 95%, about 98%, about 99%, about 99.5%, or about 99.9% purity by weight (excluding weight contributed by water, or weight contributed by a pharmaceutically acceptable carrier when formulated for administration to a patient). Thus, in one embodiment, the peptides are purified. In another embodiment, the peptides are isolated, that is, isolated from other impurities that arose during synthesis. In another embodiment, the peptides are purified and isolated.

Modifications can be made to the peptides, such as modifications to confer resistance to enzymatic degradation such as adding blocking groups to both the N- and C-terminal residues. Another method for preventing degradation and premature clearance by the renal system is the use of unnatural amino acid substitutes in the peptide sequence. For example, N-methyl-alanine is often substituted for alanine and α-amino isobutyric acid and α-amino butyric acid are substitutes for bulky hydrophobic amino acids.

Recombinant techniques, as known in the art, may also be used to produce peptides suitable for the methods disclosed herein. Naturally-occurring proteins may be cleaved to produce a desired NF κB-inhibitor. Methods of designing and screening small molecules may also be used. Methods to generate and screen peptido-mimetics may also be useful in producing NF κB-inhibitors. Substances that are a mixture of one or more peptides and one or more peptido-mimetics may also be used.

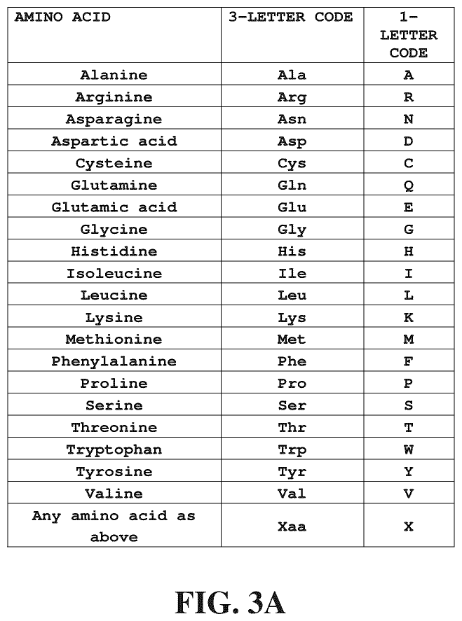

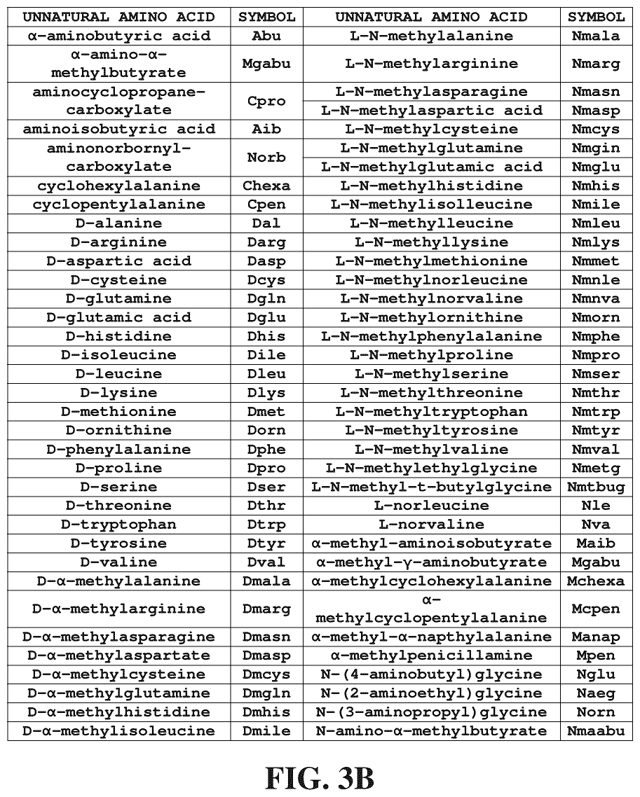

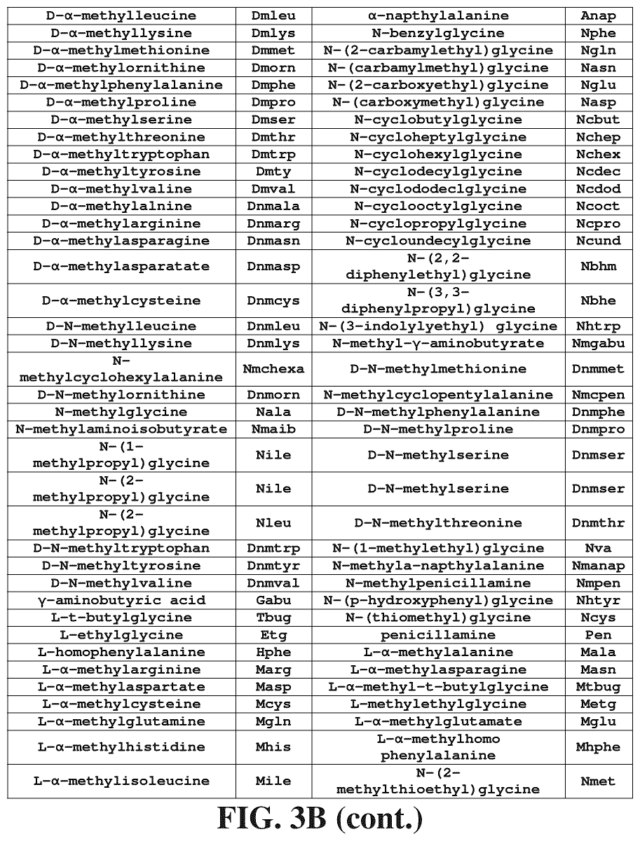

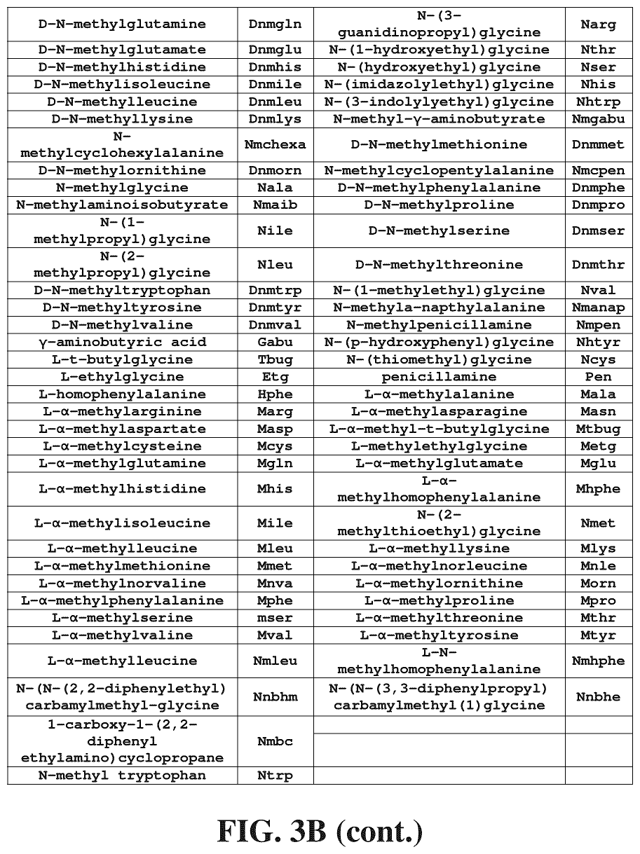

AMINO ACID RESIDUES: Amino acids and their three and one letter codes are used in their customary format; non-proteinogenic amino acids are listed using standard abbreviations. FIG. 3A lists the naturally-occurring (proteinogenic) encoded amino acids. FIG. 3B lists unnatural and/or non-encoded (non-proteinogenic) amino acids.

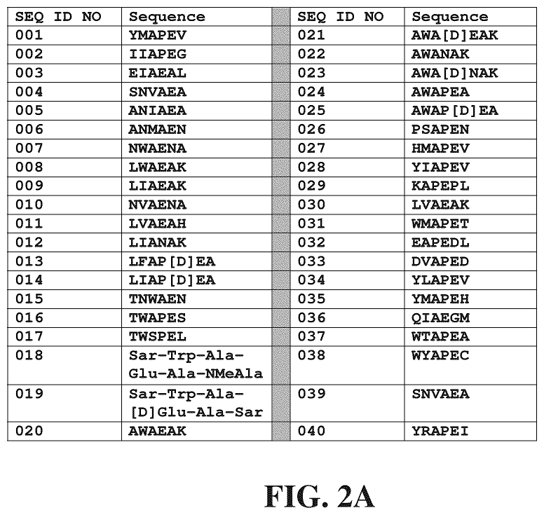

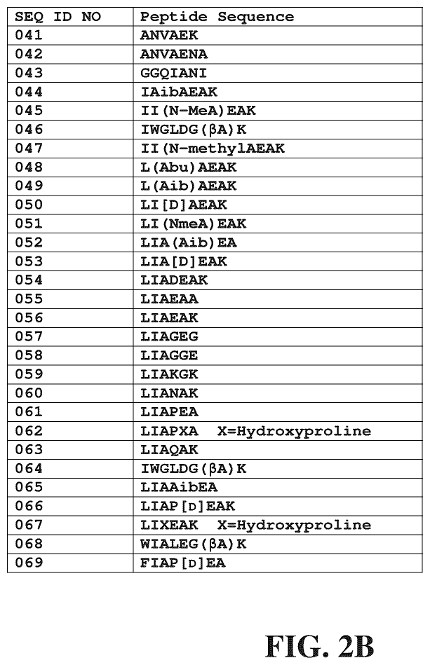

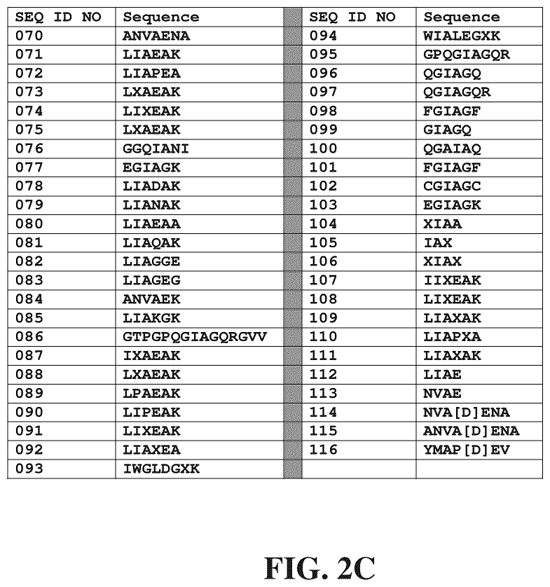

EXAMPLES OF PEPTIDES FOR USE IN THE METHODS DISCLOSED HEREIN: The list of peptides in FIG. 2A, FIG. 2B, and FIG. 2C is presented only as examples of peptides incorporating the bioactive core sequences as described herein and is not meant to be comprehensive.

FORMULATION: According to some embodiments, the present disclosure provides a pharmaceutical composition comprising, as an active ingredient, an agent capable of inhibiting NF κB activation, and a pharmaceutically acceptable carrier, excipient or diluent. As used herein, a “pharmaceutical composition” refers to a preparation of one or more of the active ingredients described herein, with other components such as physiologically suitable carriers and excipients. The purpose of a pharmaceutical composition is to facilitate administration of a compound to a subject.

A “pharmaceutically acceptable carrier” refers to a carrier or a diluent that does not cause significant irritation to an organism and does not abrogate the biological activity and properties of the administered compound. Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

In another embodiment of the present disclosure, a therapeutic composition further comprises a pharmaceutically acceptable carrier. As used herein, a “carrier” refers to any substance suitable as a vehicle for delivering of the agents or molecule of the present disclosure to a suitable in vivo or in vitro site. As such, carriers can act as a pharmaceutically acceptable excipient of a therapeutic composition of the present disclosure. Carriers of the present disclosure include: (1) excipients or formularies that transport, but do not specifically target a molecule to a cell (referred to herein as non-targeting carriers); and (2) excipients or formularies that deliver a molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Therapeutic compositions of the present disclosure can be sterilized by conventional methods.

Targeting carriers are herein referred to as “delivery vehicles”. Delivery vehicles of the present disclosure are capable of delivering a therapeutic composition of the present disclosure to a target site in a subject. A “target site” refers to a site in a subject to which one desires to deliver a therapeutic composition. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles. A delivery vehicle of the present disclosure can be modified to target to a particular site in a subject. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell type by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. For example, an antibody specific for an antigen found on the surface of a target cell can be introduced to the outer surface of a liposome delivery vehicle so as to target the delivery vehicle to the target cell. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics. This list is not comprehensive and additional formulations may be developed to deliver the peptide inhibitor of NF κB.

One or more of the peptides may be formulated as a pharmaceutical composition. Such composition may be administered topically, orally, intravenously, by inhalation, by infusion, by injection, intraperitoneally, intramuscularly, subcutaneously, intra-aurally, by intra-articular administration, by intra-mammary administration, by intra-arterial injection, rectally, by topical administration or by absorption through epithelial and/or muco-cutaneous linings. This list is not comprehensive and additional formulations may be developed to deliver the peptide inhibitor of NF κB. For treatment of systemic inflammation, intravenous administration is useful.

The peptides may be formulated such as: a solid, powder, lyophilized powder, aqueous solution, aqueous suspension, solution in an excipient liquid, emulsion, paste, spray, cream, lotion, controlled release formulation, tablet, pill, gel, liposome, on a patch, in an implant, on a tape, capsule, lozenge, dragee, gel, syrup, slurry and/or a suspension, formulated with a solid excipient, carbohydrate, protein filler, sugar such as lactose, sucrose, mannitol, sorbitol, starch, cellulose, methyl cellulose, hydroxypropylmethyl-cellulose sodium carboxy-methylcellulose, cross-linked polyvinyl pyrrolidone, gum, tragacanth, gelatin, collagen, disintegrating or solubilizing agent, agar, alginic acid alginic salt, sodium alginate. This list is not comprehensive and additional formulations may be developed to deliver the peptide inhibitor of NF κB.

The peptides in various embodiments as described above may be administered in delivery devices and systems such as slow release compositions, mechanical and electro-mechanical devices, nano-particles, microspheres, liposomes, adhesive films and pastes, mucoadhesives, oral mucoadhesives, vaginal mucoadhesives, Orajel, Orabase, pastes, solution, solid excipients, intravenously, intra-dermally, intra-muscularly, intra-peritoneally, by ultrasound and iontophoresis, by electroporation, hydrophilic emulsion foam, lipophilic emulsion foam, nanoemulsion foam, aqueous foam, hydroethanolic foam, potent-solvent foam, suspension foam, ointment foam, hydrophilic ointment foam, oil foam, saccharide foams, as part of dendrimers. This list is not comprehensive and additional formulations may be developed to deliver the peptide inhibitor of NF κB.

Therapeutic peptides can be delivered via inhalation using methods known in the art; see, e.g., Inhalation Delivery of Therapeutic Peptides and Proteins. 1st Edition (Akwete Adjei, Pramod K. Gupta, Eds.), Taylor & Francis, 1997 (Lung Biology in Health and Disease. Vol. 107, Claude Lenfant, Exec. Editor); or Drug Delivery to the Lung, (Hans Bisgaard, Chris O'Callaghan, Gerald C. Smaldone, Eds.), New York: Marcel Dekker, Inc., 2001 (Lung Biology in Health and Disease. Vol. 162, Claude Lenfant, Exec. Editor). Delivery via inhalation can be used both for local treatment of the respiratory complications of COVID-19, and for absorption into circulation and systemic delivery. The peptides, or pharmaceutically acceptable salts thereof, can be dissolved or suspended in liquids and delivered as aerosols, such as mists or sprays, to the respiratory tract, e.g., lungs, trachea, throat, etc. The peptides, or pharmaceutically acceptable salts thereof, can be prepared as fine powders and delivered via gaseous carrier to the lungs.

The peptides in various embodiments as described above may be administered in combination with and as adjuncts to therapeutic and palliative regimens and may be delivered with one or more of additives such as, analgesics, antibiotics, antivirals, growth factors, palliatives, anti-oxidants, vitamins, MMP-inhibitors. This list is not comprehensive and additional formulations may be developed to deliver the peptide inhibitor of NF κB.

In addition to the formulations described above, the compounds (that is, the disclosed peptides as described herein) can be formulated as pharmaceutical compositions by formulation with additives such as pharmaceutically acceptable excipients, pharmaceutically acceptable carriers, and pharmaceutically acceptable vehicles. Suitable pharmaceutically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), incorporated herein by reference.

A pharmaceutical composition can comprise a unit dose formulation, where the unit dose is a dose sufficient to have a therapeutic or suppressive effect on a disease or disorder. The unit dose may be sufficient as a single dose to have a therapeutic or suppressive effect on a disease or disorder. Alternatively, the unit dose may be a dose administered periodically in a course of treatment or suppression of a disease or disorder.

Pharmaceutical compositions containing the compounds of the present disclosure and compositions containing the compounds of the present disclosure may be in any form suitable for the intended method of administration, including, for example, a solution, a suspension, or an emulsion. Liquid carriers are typically used in preparing solutions, suspensions, and emulsions. Liquid carriers contemplated for use in the practice of the present disclosure include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soy bean oil, coconut oil, olive oil, safflower oil, cottonseed oil, and the like. For parenteral administration, the carrier can also be an oily ester such as ethyl oleate, isopropyl myristate, and the like. Compositions of the present disclosure may also be in the form of microparticles, microcapsules, liposomal encapsulates, and the like, as well as combinations of any two or more thereof.

Time-release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly (phosphoesters), polyamides, polyurethanes, poly (imidocarbonates) and poly (phosphazenes).

The compounds disclosed herein may be administered enterally, orally, parenterally, sublingually, by inhalation (e.g. as mists or sprays), rectally, or topically in dosage unit formulations containing conventional nontoxic pharmaceutically acceptable carriers, adjuvants, and vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, and the like, and directly to a specific or affected organ or tissue. Microinjection can also be utilized, as well as needle-free injection, such as jet injection. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intrasternal injection, or infusion techniques. The compounds are mixed with pharmaceutically acceptable carriers, adjuvants, and vehicles appropriate for the desired route of administration. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

In some embodiments disclosed herein, especially those embodiments where a formulation is administered by inhalation, injection, or other parenteral administration including the routes listed herein, but also including embodiments used for oral, gastric, gastrointestinal, or enteric administration, the formulations and preparations used in the methods disclosed herein are sterile. Methods for preparing sterile, pharmaceutically acceptable compositions include steam sterilization, dry-heat sterilization, gas sterilization, ionizing radiation, or sterile filtration. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present disclosure can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present disclosure, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

The disclosure also provides articles of manufacture and kits containing materials useful for treating or suppressing diseases or disorders. The disclosure also provides kits comprising any one or more of the peptides disclosed herein. In some embodiments, the kit comprises printed instructions for use of the peptide or peptides, such as instructions for formulation, sterilization, or administration. In other aspects, the kits may be used for any of the methods described herein.

The amount of active ingredient that may be combined with the carrier materials to produce a single dosage form will vary depending upon the host to which the active ingredient is administered and the particular mode of administration. It will be understood, however, that the specific dose level for any particular patient will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The pharmaceutical unit dosage chosen is usually fabricated and administered to provide a defined final concentration of drug in the blood, tissues, organs, or other targeted region of the body. The therapeutically effective amount or effective amount for a given situation can be readily determined by routine experimentation and is within the skill and judgment of the ordinary clinician.

Examples of dosages which can be used are a therapeutically effective amount or effective amount within the dosage range of about 0.1 mg/kg to about 300 mg/kg body weight, or within about 1.0 mg/kg to about 100 mg/kg body weight, or within about 1.0 mg/kg to about 50 mg/kg body weight, or within about 1.0 mg/kg to about 30 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Compounds of the present disclosure may be administered in a single daily dose, or the total daily dosage may be administered in divided dosage of two, three or four times daily.

While the compounds disclosed herein can be administered as the sole active pharmaceutical agent, or as the sole active pharmaceutical agent present in a therapeutically effective amount, they can also be used in combination with one or more other agents used in the treatment or suppression of diseases or disorders. When additional active agents are used in combination with the compounds of the present disclosure, the additional active agents may generally be employed in therapeutic amounts as indicated in the Physicians Desk Reference (PDR) 53rd Edition (1999), or such therapeutically useful amounts as would be known to one of ordinary skill in the art.

The compounds disclosed herein and the other therapeutically active agents can be administered at the recommended maximum clinical dosage or at lower doses. Dosage levels of the active compounds in the compositions disclosed herein may be varied so as to obtain a desired therapeutic response depending on the route of administration, severity of the disease and the response of the patient. When administered in combination with other therapeutic agents, the therapeutic agents can be formulated as separate compositions that are given at the same time or different times, or the therapeutic agents can be given as a single composition.

DISEASES: Some of the diseases in which the therapeutic compounds and compositions disclosed herein are indicated are specific NF κB associated diseases, that is to say, diseases caused by the unwanted activation of genes under control of the transcriptional regulatory factor NF κB. Accordingly, the diseases disclosed herein can be treated with the disclosed peptides, including one or more peptides selected from the group consisting of CORE SEQUENCE (1), CORE SEQUENCE (1-N), CORE SEQUENCE (1-U), GROUP (1-S), GROUP (1-T), GROUP (1-V), GROUP (1-W), CORE SEQUENCE (2), CORE SEQUENCE (2-N), CORE SEQUENCE (2-U), GROUP (2-S), GROUP (2-T), GROUP (2-V), CORE SEQUENCE (3), CORE SEQUENCE (3-N), CORE SEQUENCE (3-U), GROUP (3-S), GROUP (3-T), GROUP (3-V), GROUP (3-W), CORE SEQUENCE (4), CORE SEQUENCE (4-N), CORE SEQUENCE (4-U), GROUP (4-S), GROUP (4-T), GROUP (4-V), CORE SEQUENCE (5), CORE SEQUENCE (5-N), CORE SEQUENCE (5-U), GROUP (5-S), GROUP (5-T), GROUP (5-V), GROUP (5-W), CORE SEQUENCE (6), CORE SEQUENCE (6-N), CORE SEQUENCE (6-U), GROUP (6-S), GROUP (6-T), GROUP (6-V), CORE SEQUENCE (7), CORE SEQUENCE (7-N), CORE SEQUENCE (7-U), GROUP (7-S), GROUP (7-T), GROUP (7-V), GROUP (7-W), CORE SEQUENCE (8), CORE SEQUENCE (8-N), CORE SEQUENCE (8-U), GROUP (8-S), GROUP (8-T), GROUP (8-V). SEQ ID NOS:001-069. (SEQ ID NOS:001-008, 010, 011, and 013-040), and (SEQ ID NOS:001-007, 010, 011, 013-029, and 031-040).

GENE REGULATION IN COVID-19: Therapeutic intervention in patients suffering from COVID-19 or other inflammatory diseases will often result in significant changes in gene expression in affected tissues. Downregulation of genes involved in the inflammatory response is highly desirable. In addition. SARS-COV-2 enters cells via the ACE2 receptor, and downregulation of ACE2 receptor expression can aid in treatment of COVID-19 by reducing the infection of cells. TMPRSS2 also contributes to viral infectivity and activation, and downregulation of TMPRSS2 can also aid in treatment of COVID-19. Reducing the expression of CHUK, which catalyzes the final step in NF kappa B (NF-κB) activation, is also beneficial in COVID-19 patients or patients suffering from other inflammatory diseases, as is reducing expression of subunits of NF-κB, such as NFKB1 or NFKB2. Cytokines, interleukins, chemokines, or their receptors play a major role in inflammation and in the pathologies associated with COVID-19. TNF is a cytokine induced by pathogens, and TNERSF1A is the primary receptor for TNF. TNFRSF9 is induced by T-cell activation. IL1A is induced by pathogens. IL1B is a mediator of inflammation. IL6 and CXCL8 induce acute phase proteins. CCL2 is a cytokine induced by pathogens. CCL5 is another mediator of inflammation. All of these genes contribute to NF-κB activation or other undesirable effects, and downregulation of their expression is beneficial.

Disclosed herein are methods of treating COVID-19 in a patient in need thereof, comprising administering to the patient an amount of one or more peptides effective to reduce expression of a gene, where expression of said gene promotes inflammation or contributes to the activity of NF kappa B. In one embodiment, disclosed herein is a method of treating COVID-19 in a patient in need thereof, comprising administering to the patient an amount of one or more peptides effective to reduce expression of ACE2 receptor. In one embodiment, disclosed herein is a method of treating COVID-19 in a patient in need thereof, comprising administering to the patient an amount of one or more peptides effective to reduce expression of TMPRSS2. In one embodiment, disclosed herein is a method of treating COVID-19 in a patient in need thereof, comprising administering to the patient an amount of one or more peptides effective to reduce expression of both ACE2 receptor and TMPRSS2. In one embodiment, disclosed herein is a method of treating COVID-19 in a patient in need thereof, comprising administering to the patient an amount of one or more peptides effective to reduce expression of CHUK.

In one embodiment, disclosed herein is a method of treating COVID-19 in a patient in need thereof, comprising administering to the patient an amount of one or more peptides effective to reduce expression of NFKB1. In one embodiment, disclosed herein is a method of treating COVID-19 in a patient in need thereof, comprising administering to the patient an amount of one or more peptides effective to reduce expression of NFKB2. In one embodiment, disclosed herein is a method of treating COVID-19 in a patient in need thereof, comprising administering to the patient an amount of one or more peptides effective to reduce expression of NFKB1 and of NFKB2.

In one embodiment, disclosed herein is a method of treating COVID-19 in a patient in need thereof, comprising administering to the patient an amount of one or more peptides effective to reduce expression of TNF. In one embodiment, disclosed herein is a method of treating COVID-19 in a patient in need thereof, comprising administering to the patient an amount of one or more peptides effective to reduce expression of TNFRSF1A. In one embodiment, disclosed herein is a method of treating COVID-19 in a patient in need thereof, comprising administering to the patient an amount of one or more peptides effective to reduce expression of TNFRSF9. In one embodiment, disclosed herein is a method of treating COVID-19 in a patient in need thereof, comprising administering to the patient an amount of one or more peptides effective to reduce expression of TNF, TNFRSF1A, and TNFRSF9.

In one embodiment, disclosed herein is a method of treating COVID-19 in a patient in need thereof, comprising administering to the patient an amount of one or more peptides effective to reduce expression of IL1A. In one embodiment, disclosed herein is a method of treating COVID-19 in a patient in need thereof, comprising administering to the patient an amount of one or more peptides effective to reduce expression of IL1B. In one embodiment, disclosed herein is a method of treating COVID-19 in a patient in need thereof, comprising administering to the patient an amount of one or more peptides effective to reduce expression of IL6. In one embodiment, disclosed herein is a method of treating COVID-19 in a patient in need thereof, comprising administering to the patient an amount of one or more peptides effective to reduce expression of IL1A, IL1B, and IL6.

In one embodiment, disclosed herein is a method of treating COVID-19 in a patient in need thereof, comprising administering to the patient an amount of one or more peptides effective to reduce expression of CCL2. In one embodiment, disclosed herein is a method of treating COVID-19 in a patient in need thereof, comprising administering to the patient an amount of one or more peptides effective to reduce expression of CCL5. In one embodiment, disclosed herein is a method of treating COVID-19 in a patient in need thereof, comprising administering to the patient an amount of one or more peptides effective to reduce expression of CXCL8. In one embodiment, disclosed herein is a method of treating COVID-19 in a patient in need thereof, comprising administering to the patient an amount of one or more peptides effective to reduce expression of CCL2, CCL5, and CXCL8.

In any of the disclosed embodiments, gene expression can be reduced by at least about 25%, as compared to gene expression prior to treatment. In any of the disclosed embodiments, gene expression can be reduced by at least about 50%, as compared to gene expression prior to treatment. In any of the disclosed embodiments, gene expression can be reduced by at least about 75%, as compared to gene expression prior to treatment. In any of the disclosed embodiments, gene expression can be reduced by at least about 90%, as compared to gene expression prior to treatment.

In any of the disclosed embodiments, the one or more peptides administered comprises ANVAENA.

In any of the disclosed embodiments, the one or more peptides administered comprises YMAP[D]EV.

In any of the disclosed embodiments, the one or more peptides administered comprises a combination of ANVAENA and YMAP[D]EV.

### EXAMPLES

The following examples are provided to illustrate, but not limit, the invention.

### Example 1

### In Vitro Screening of Peptide Activity: Gene Expression Studies in an In Vitro Model of Vascular Endothelium

Gene expression in the presence of peptides. Peptides are shown to inhibit the production of inflammatory proteins in a model for vascular endothelium. Human umbilical vein endothelial cells are obtained from ATCC (American Type Culture Collection). Human umbilical vein endothelial cells are grown in tissue culture medium consisting of M-199 (Gibco, Grand Island, N.Y.) supplemented with 10% fetal bovine serum, 10% defined bovine calf serum (Gemini Bio-Products, Inc., Calabasas, Calif.), and 2 mM L-glutamine with penicillin and streptomycin (Irvine Scientific). The cells are grown in multiwall tissue culture plates (Falcon, Lincoln Park, N. J.) coated with fibronectin (Collaborative Biomedical Products, Bedford, Mass.). All experiments are performed using tightly confluent third-passage endothelial cells. Cell are grown to approximately 5×10⁴ endothelial cells per cm² of culture surface at confluency.

Endothelial cell stimulation. On the day of the experiment, the tissue culture medium above the endothelial cells is aspirated and replaced with fresh medium containing 10 nanogram/ml of TNF-alpha. The following sets of cultures are used to determine the inhibitory role of peptide in inflammation:
1. Baseline Control: No treatment.
2. Effect of the peptide on normal cells: control cells are treated with an individual peptide, or a combination of peptides, such as a combination of two peptides.
3. Inflammation Model, no peptide treatment: cells are treated with TNF-alpha.
4. Inflammation Model with peptide treatment: peptide or combination of peptides are added to TNF-alpha cultures 1 hour after TNF-alpha treatment. Cells are evaluation to determine extent to which peptide (or peptides) blocks TNF-alpha induced inflammation and production of inflammatory proteins.

Cells are harvested after 24 hours, and RNA is extracted using a commercial kit (Qiagen). Gene microarray studies are carried out using Affymetrix gene chips or Cellecta (Mountain View, California) DriverMap™ gene analysis.

Peptides can be added at concentrations of 10 micromolar of an individual peptide. A combination of two peptides can be added with each peptide at 5 micromolar concentration. 10 micromolar YMAP[D]EV, 10 micromolar ANVAENA, or a combination of 5 micromolar YMAP[D]EV and 5 micromolar ANVAENA can be added to test peptides.

### Example 2

### In Vivo Screening of Peptide Activity: Paw Edema Model

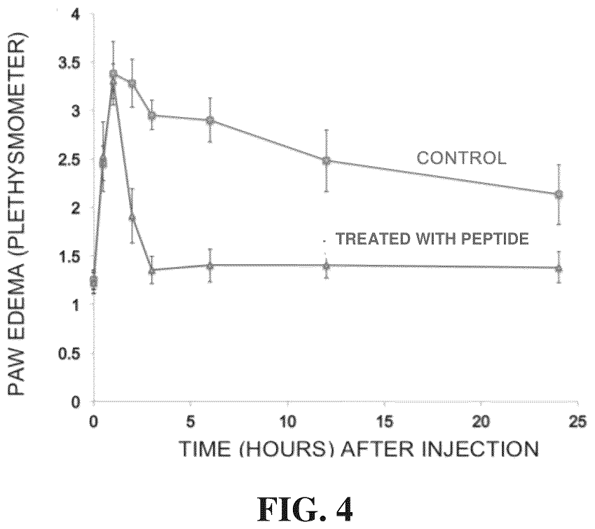

The anti-inflammatory activity of YMAP[D]EV was also examined in a classical carrageenan (CAR) induced inflammation model (Ivan G. Otterness, Peter F. Moore, Carrageenan foot edema test, Methods in Enzymology, Academic Press, Volume 162, 1988, Pages 320-327, (see URL www.sciencedirect.com/science/article/pii/0076687988620866); Fehrenbacher J C et al., Models of inflammation: Carrageenan- or complete Freund's Adjuvant (CFA)-induced edema and hypersensitivity in the rat. Curr Protoc Pharmacol. 2012 March; Chapter 5m Unit5.4, doi: 10.1002/0471141755.ph0504s56. PMID: 22382999; PMCID: PMC4683998). Paw edema was induced by a sub-plantar injection of CAR (100 μL of a 1% suspension in 0.85% saline). In the treatment group, YMAP[D]EV (70 ng, 1 nanomole) was injected 30 minutes after CAR injection. Changes in paw volume were measured using a plethysmometer immediately before CAR injection, and then at hourly intervals for 24 h. Edema was expressed as change in paw volume (mL) after CAR injection relative to the pre-injection value for each rat. As seen in FIG. 4, abrogation of inflammation was rapid and long lasting. There was a marked decrease in edema within two hours, and the effect lasted through the 24-hour period of observation.

Similar studies were carried out with ANVAENA, and yielded nearly identical data. The combination of YMAP[D]EV and ANVAENA should thus be a useful anti-inflammatory drug in humans suffering with COVID-19.

### Example 3

### Differential Gene Expression Data Shows Complementary Action of Combination of ANVAENA (PEP1) and YMAP[D]EV (PEP 2)

Gene expression data was generated by the procedure described in Example 1 for ANVAENA (PEPTIDE 1) (Affymetrix gene arrays) and YMAP[D]EV (PEPTIDE 2)) (DriverMap™ gene analysis). Expression of genes important in the disease process of COVID-19 and other inflammatory disorders was examined. The tables below show the RNA expression of genes in cells treated with the peptides as indicated versus control cells. For example, in Table A, treatment of cells in the procedure described in Example 1 with PEP1 (ANVAENA) resulted in reduced expression of ACE2 receptor compared to control cells. The numbers are expressed as ratios of treated cells to control cells; e.g., the amount of ACE2 receptor RNA expressed in cells treated with PEP1 normalized to the amount of ACE2 receptor expressed is 0.632 (or 63.2%) expression of ACE2 receptor in cells treated with PEP 1 compared to control cells, set at 1.000 (or 100%). Numbers in bold font are desirable results for treatment of COVID-19 and other inflammatory disorders.

A. Key Proteins In Viral Entry: Angiotensin-converting enzyme 2 receptor (ACE2 receptor) provides the primary entry point for the SARS-COV-2 virus into cells, while TMPPRS2 proteolytically facilitates viral entry in cells. Downregulating ACE2 receptor expression and TMPPRS2 reduces the susceptibility of cells to SARS-COV-2 infection.

TABLE A

| GENE | PEP1 | PEP2 | ROLE IN COVID-19 |
|---|---|---|---|
| ACE2 | 0.632 | 1.000 | ACE2 receptor is the primary receptor that |
| receptor | | | binds virions. TMPPRS2 proteolytically |
| TMPRSS2 | 0.525 | 1.000 | facilitates viral entry in cell. |

B. Viral Entry Into Cell Activates NF kappa B: Activated NF kappa B transcription factor turns on the innate immune system, which runs rampant in COVID-19. Increased NF kappa B generates cytokines and many harmful proteins that cause fever, pulmonary dysfunction, vascular dysfunction and tissue breakdown. Further, many target genes of NF kappa B activate NF kappa B, initiating a self-amplifying feedback cycle that contributes to the severity and morbidity of COVID-19. As shown in Table B, PEPTIDE 1 and PEPTIDE 2 differentially affect the expression of components of the NF kappa B pathway, reducing the deleterious effects of COVID-19.

TABLE B

| GENE | PEP1 | PEP2 | ROLE IN COVID-19 |
|---|---|---|---|
| CHUK | 0.068 | 0.937 | CHUK catalyzes the final step in |
| | | | NF-κB activation |
| NFKB1 | 0.927 | 0.243 | Important subunits of NF-κB that on |
| NFKB2 | 0.851 | 0.138 | activation enter the nucleus and bind |
| | | | to DNA causing the expression of |
| | | | numerous undesirable genes |
| NFKBIA | 1.22 | 0.144 | Indicator of NF-κB activation process. |

C. and D. Cytokines, Interleukins, and Their Receptors: Cytokines and interleukins play a major role in inflammation and in the exacerbation of morbidities associated with COVID-19.

TABLE C

| GENE | PEP1 | PEP2 | ROLE IN COVID-19 |
|---|---|---|---|
| TNF | 0.349 | 0.015 | Cytokine induced by pathogens. |
| | | | Induces NF-κB activation. |
| TNFRSF1A | 1.415 | 0.752 | Primary receptor for TNF |
| | | | leading to NF-κB activation. |
| TNFRSF9 | 1.136 | 0.007 | Induced by T-cell activation, |
| | | | promotes NF-κB activation. |

TABLE D

| GENE | PEP1 | PEP2 | ROLE IN COVID-19 |
|---|---|---|---|
| ILIA | 0.036 | 0.260 | Cytokine induced by pathogens. |
| | | | Induces NF-κB activation. |
| IL1B | 0.691 | 0.010 | Mediator of inflammation, |
| | | | promotes NF-κB activation |
| IL6 | 1.135 | 0.057 | Induces acute phase proteins. |

E. Chemokines differentially affected by Peptide 1 and Peptide 2. These proteins are associated with viral infection.

TABLE E

| GENE | PEP1 | PEP2 | ROLE IN COVID-19 |
|---|---|---|---|
| CCL2 | 1.298 | 0.059 | Cytokine induced by pathogens. |
| | | | Induces NF-κB activation. |
| CCL5 | 0.456 | 0.0003 | Mediator of inflammation, |
| | | | promotes NF-κB activation |
| CXCL8 | 1.135 | 0.0052 | Induces acute phase proteins. |

As can be seen above, ANVAENA (PEPTIDE 1) and YMAP[D]EV (PEPTIDE 2) contribute to the reduced expression of genes that are harmful in COVID-19. Importantly, the two peptides complement each other. For several of the genes studied, where one of the peptides may not downregulate expression of a particular gene, the other peptide will. For example, in Table B, PEP 1 strongly downregulates CHUK, while PEP2 has little effect. Conversely, PEP1 has little effect on NFKBIA expression (and indeed, upregulates the gene somewhat), while PEP2 strongly downregulates it. While either peptide can be used alone to treat COVID-19, use of the two peptides in combination leads to improved treatment.

### Example 4

### In Vivo Testing of Peptides in Animals

Activity of peptides against COVID-19 can be tested in experimental animals. Kim, Young-Il et al., “Infection and Rapid Transmission of SARS-COV-2 in Ferrets,” Cell Host Microbe 27(5): 704-709 (2020) have described a ferret model for COVID-19. The K18-hACE2 mouse model developed by McCray P. B. et al. (“Lethal infection of K18-hACE2 mice infected with severe acute respiratory syndrome coronavirus,” J Virol. 81(2):813-821 (2007)) for study of the earlier SARS coronavirus, SARS-COV, has also been used to study SARS-COV-2: see Bao L. et al., “The pathogenicity of SARS-COV-2 in hACE2 transgenic mice,” Nature 583:830-833 (2020); McCray P. B. et al. Macaca mulatta has been identified as a suitable non-human primate model for SARS-COV-2 infection; see Lu, S. et al., “Comparison of nonhuman primates identified the suitable model for COVID-19,” Signal Transduct Target Ther. 5: 157 (2020). Experiments are carried out in accordance with institutional policies for animal studies.

Experimental animals are inoculated with SARS-COV (for example, by nasal inoculation). Biological samples, including nasal washes or nasal swabs, saliva, urine, feces, and blood are collected and viral load is quantitated. After onset of symptoms, including fever, lung congestion, or cytokine storm, peptides are administered to one group of animals, while placebo or vehicle is administered to a control group of animals. Peptides, or placebo or vehicle, can be administered intravenously: or if desired, an alternate route of administration can be selected. The disease course in both groups is monitored in order to demonstrate the efficacy of treatment, including reduction of inflammation, reduction of cytokine, interleukin, or chemokine levels, and overall recovery and time to recovery from the disease.

### Example 5

### Administration of Peptides to Patients

Clinical trials are carried out in accordance with all applicable legal and ethical requirements and in accordance with institutional policies. Peptides are administered intravenously to patients suffering from COVID-19 (if desired, other routes of administration can be used). Comparison to clinical outcomes using other modalities, such as remdesivir, is conducted.

The disclosures of all publications, patents, patent applications and published patent applications referred to herein by an identifying citation are hereby incorporated herein by reference in their entirety. Web sites references using “World-Wide-Web” at the beginning of the Uniform Resource Locator (URL) can be accessed by replacing “World-Wide-Web” with www.

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

1. A composition for treating COVID-19 comprising a therapeutically effective amount of a combination of:
i) a first peptide of length six to twenty amino acids comprising the sequence YMAP[D]EV; and
ii) a second peptide of length seven to twenty amino acids comprising the sequence ANVAENA.

2. The composition of claim 1, wherein the first peptide is YMAP[D]EV and the second peptide is ANVAENA.

3. The composition of claim 1, further comprising a pharmaceutically acceptable carrier; or
wherein the composition is formulated for intravenous administration; or
wherein the composition is formulated for intraperitoneal administration; or
wherein the composition is formulated for administration to the lungs.

4-6. (canceled)

7. A method of treating COVID-19 in a patient in need thereof, comprising administering to a patient in need thereof the composition of claim 1.

8. (canceled)

9. A method of treating COVID-19 in a patient in need thereof, comprising administering to a patient in need thereof a composition comprising a therapeutically effective amount of a peptide of length six to twenty amino acids comprising the sequence YMAP[D]EV.

10. The method of claim 9, wherein the peptide is YMAP[D]EV.

11. A method of treating COVID-19 in a patient in need thereof, comprising administering to a patient in need thereof a composition comprising a therapeutically effective amount of a peptide of length seven to twenty amino acids comprising the sequence ANVAENA.

12. The method of claim 11, wherein the peptide is ANVAENA.

13. The method of claim 7, wherein the patient is suffering from one or more of inflammation, fever, pneumonia, hypercytokinemia, acute respiratory distress syndrome, edema, coagulation, or lymphopenia.

14-17. (canceled)

18. A method of treating COVID-19 in a patient in need thereof, comprising administering to a patient in need thereof a composition comprising a therapeutically effective amount of one or more peptides of four to twenty amino acid residues in length, wherein the peptide inhibits the activity of NF-kappa B.

19-20. (canceled)

21. The method of claim 18, wherein the one or more peptides are selected from peptides comprising the sequence:
CORE SEQUENCE (1): Xxx-Ala-Pro-Glu
where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala;
CORE SEQUENCE (2): Xxx-Ala-Pro-D-Glu
where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala;
CORE SEQUENCE (3): Xxx-Ala-Glu-Ala
where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala;
CORE SEQUENCE (4): Xxx-Ala-D-Glu-Ala
where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala;
CORE SEQUENCE (5): Xxx-Ala-Asn-Ala
where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala;
CORE SEQUENCE (6): Xxx-Ala-D-Asn-Ala
where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala;
CORE SEQUENCE (7): Xxx-Ala-Glu-Asn
where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala;
CORE SEQUENCE (8): Xxx-Ala-D-Glu-Asn
where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala;
SEQ ID NOS:001-069; SEQ ID NOS:001-008, 010, 011, and 013-040; SEQ ID NOS:001-007, 010, 011, 013-029, and 031-040; and SEQ ID NO:116;
wherein the one or more peptides contains at least one amino acid selected from the group consisting of D-amino acids, unnatural amino acids, and non-proteinogenic amino acids.

22. The method of claim 18, wherein the one or more peptides are selected from peptides comprising the sequence: CORE SEQUENCE (1), CORE SEQUENCE (1-N), CORE SEQUENCE (1-U), GROUP (1-S), GROUP (1-T), GROUP (1-V), GROUP (1-W), CORE SEQUENCE (2), CORE SEQUENCE (2-N), CORE SEQUENCE (2-U), GROUP (2-S), GROUP (2-T), GROUP (2-V), CORE SEQUENCE (3), CORE SEQUENCE (3-N), CORE SEQUENCE (3-U), GROUP (3-S), GROUP (3-T), GROUP (3-V), GROUP (3-W), CORE SEQUENCE (4), CORE SEQUENCE (4-N), CORE SEQUENCE (4-U), GROUP (4-S), GROUP (4-T), GROUP (4-V), CORE SEQUENCE (5), CORE SEQUENCE (5-N), CORE SEQUENCE (5-U), GROUP (5-S), GROUP (5-T), GROUP (5-V), GROUP (5-W), CORE SEQUENCE (6), CORE SEQUENCE (6-N), CORE SEQUENCE (6-U), GROUP (6-S), GROUP (6-T), GROUP (6-V), CORE SEQUENCE (7), CORE SEQUENCE (7-N), CORE SEQUENCE (7-U), GROUP (7-S), GROUP (7-T), GROUP (7-V), GROUP (7-W), CORE SEQUENCE (8), CORE SEQUENCE (8-N), CORE SEQUENCE (8-U), GROUP (8-S), GROUP (8-T), GROUP (8-V), SEQ ID NOS:001-069, (SEQ ID NOS:001-008, 010, 011, and 013-040), (SEQ ID NOS:001-007, 010, 011, 013-029, and 031-040), and (SEQ ID NO:116).

23. The method of claim 18, wherein the one or more peptides are selected from peptides comprising the sequence:
CORE SEQUENCE (2): Xxx-Ala-Pro-D-Glu
where Xxx is selected from the group consisting of Met, Ile, Val, Cys, Trp, Tyr, Phe, 5-methyl-Trp, allo-Ile, β-styryl-Ala, naphthyl-Ala, diphenyl-Ala, α-aminobutyric acid, α-aminocaproic acid, norleucine, α-amino-2-phenylbutyric acid, α-Amino-1-naphthalenepropanoic acid, β-cyclohexyl-Ala, dehydroalanine, and β-tert-butyl-Ala.

24-40. (canceled)

41. The method of claim 18, wherein the patient is suffering from multisystem inflammatory syndrome in children (MIS-C).

42. A method of treating COVID-19 in a patient in need thereof, comprising administering to the patient an amount of one or more peptides effective to reduce expression of ACE2 receptor, TMPRSS2, or both ACE2 receptor and TMPRSS2; or
administering to the patient an amount of one or more peptides effective to reduce expression of at least one of CHUK, NFKB1, or NFKB2; or
administering to the patient an amount of one or more peptides effective to reduce expression of at least one of TNF, TNFRSF1A, or TNFRSF9; or
administering to the patient an amount of one or more peptides effective to reduce expression of at least one of IL1A, IL1B, or IL6; or
administering to the patient an amount of one or more peptides effective to reduce expression of at least one of CCL2, CCL5, or CXCL8.

43-46. (canceled)

47. The method of claim 42, wherein the one or more peptides comprise ANVAENA; or
wherein the one or more peptides comprise YMAP[D]EV; or
wherein the one or more peptides comprise ANVAENA and YMAP[D]EV.

48. The method of claim 42, wherein the one or more peptides comprise YMAP[D]EV.

49. The method of claim 42, wherein the one or more peptides comprise ANVAENA and YMAP[D]EV.

50. The method of claim 42, wherein the treatment reduces expression by at least 25% relative to expression prior to treatment.

51-57. (canceled)
